SEQUENCE LISTING (12 sequences)

SEQ ID NO: 1 - DNA, 69043 bp, Homo sapiens
  exon (1204)..(1230): EXON 1 including coding region only. Non-coding portion of exon begins at 1001
catcaatcctaccttactactttcacgggattggttaaagttagcttttaaagtattttctcactctggctccttaataccctagctacagtctgacacaagaatgactgcagcaaaatgttggctgtattataataaaaatatttgtggaaaggctttgttagctggaagccatctcaagtattattctttataccccaatctcgatttaggcctctaggtaaccaaattcaaagtaacacctcatcatcatgatggttgccaaggggtaggtggagggactgcagccccttccccacaaacaattccaataaaaacactgttgtgcaaacagagcttcacgaccagttccctgggccagctgctcaccagattaccactcccaaactcccttttctttggaaatactgcacctgctgtatttgtggggagctgtataacttttgaagtatctaagtgatcagttcctacaatgacatccttgagatatggccacggaactttccaaagcagccttggcctccttcatgtccagcaacctgagataaggccacgccaccggctaagagttccgccaggggcccagctctcaggaggcctcttcggtgccgccagcctcccgaagtcatccaaggtagcaggcaaatccggcagcagcaaactagctcggattcggcttcggtttttctcctggccggcgccacaggcccctcctccggggcgggctcctcggtgcgagggcaggaggggcggagccggtggatgccgcgggctcctcctcccaagggcgggccgcgggagacccggccggctttgccggcggggaccgggctgggggcggggccgggcgccgcgagcccctcctctcgagggccggccggtttggcggcggctgcggagggtatctgaggctcggctgccaagccgaacgggctctggctcctcccagtggccggccggggcggaagcaggaggcgggggcagcgtgcgcgctgctggtcttctctcccgcggcgctggggcccgcgctccgctgctgttgctccattcggcgcttttctggcggctggctcctctccgctgccggctgctcctcgaccaggcctccttctcaacctcagcccgcggcgccgacccttccggcaccctcccgccccgtctcgtactgtcgccgtcaccgccgcggctccggccctggccccgatggctctgtgcaacggagactccaaggtcagcgtgggggtccctgcagcaccgcatcccggcggaggcgaggctcccggaccggggagccaccccgcgaggcccttccccacccccttcccccagcccgtccgcgcagccctgcgccccgggcagccacgcgtcgtagagtccctggtcggggcctgtggcatgttttatctcgggagcggcgatgctttgttgatcttgtctgcgtgtcgaggtccgggcgtcggggagggcatcagctccccgcggccgcgtcgctggcccccgcctcccccgcgccgctgtggggcgcagagcccgcgagcgcggcgcgagggtgggggtagggcggcgagcgggcttgtgtgtctgcgtgtcggggtgggggcttcctcggctcccaccccgtccctctgggaggctgggggcccgttgtggcgatcccggggcccgccgaaggcagggcagggtcggggcctcctgtgcggcgttggagctgggccggcctctggtcttcccgccaccccgtttcccgcctgccctcggcgaggcccgctccattccccccgccagccttgggctccgcggcggttaggccggttctggacgaacccctcggagctgcactgtcttccccctcccccaacgcccctgcaggcccgcgtgagggctggtggccccagggtcgcgcactccgttctccctccccgcttccttctcagagaggaaagggtgtgtgtccaggtcccgcccccgtccagcttgctccctgtcgagcttgctggtttgcggggtaacccgcttccccgcctttggggtccctccccgggacgcgagccgtgccagctcgcccgaggcctgggcagtccggagttccggagtgctggaaaagttgccgccttgacgtacccggcggggagagcacgtttgaccgctgctgctcatctctcctccaactcacttttcttagaccgttcctcttactttcgtcccgcttttgaattgggcaccttcgctttcacctctgtattaaaattttcattggcctggagttctttctgaccttcactaagtcagttttgccagcgggaaaccgtgcagttttgaattgcgatttacttcattaagatacacctcctctgtacgctgttgagggcctagctttaggaaaaaacttaattaaatttcattcggggggtgggattgtatacttcgaggtggatttggtgctctacgtcagatagtagtgccttgttcaggcagaattttaacattctttttaaaaggtgctgctcaagggtcacatcttaggaggtcctcttgaatggttgcttagcacccggtaatcattcaaaaagtatttgtcgaacaatgagtaaaagattataaggtagctgattttaactcttttaaaccaaggcaccagtggtgcttaggtccagggagctgttaagacacacaaacctttagtttctgacctaaggagcaaatgtaaaccccattgtccagctctttcccgattactagtcgacttgcaaagcatttttctgcctagttttgagtcttacaactcattggaaagattcatctttatgctgatagatgaaataactggtgttagaaggactgaaatttaatttatgggctttgaaaaatatatagtgcatttgagaggagtattttcacaaaactaagtgacagaaaagtataagatttgtttccagagtatataagaggatttttctttttcctaactggtattttttctttgttttgctctagttggaatgacaaagataatcttatcagtttgctatatttgagcctagggtgaattaattttatttcaaggttctttatgttttatagcataattaaacattttaaaaactatgttaacaattagttgtggactgaattggaagcattttcaagaggaacatctggagaattaatatatttcatcaaatctatgattattgtatgtacccctgaggaagaaagtcctgacaaaccataacatgccagtgatcataaacattttaagtaaagagatattaaatatggggaaataccttatatcacctactttattatgcatatttctaagaggcccacctggaaataagtaggagtcttattttccatagcctaccactgaaccacctttcccttgggaggaaaggcttactttgtacattgattgggccttggctgtgtgtatcttagttcataaattaaaagttgggcaagagctggttttgattcctgtttctggtacatttcagttatcaggattttctaaaacggcgtttttgcatgatagaaactggagaggggcctaaaagctatgaaagcatttgggttattttaattattttttatacacaatattttatttaatttttaatttatattatttttttgagacaggatcttgctctgtcacccaagctggagtgcagtggtgtgatcacagctcactgctgccttgacttctttagcctcctgagtagctggaagtacaggcgtggatcaccccgccctgctaatttttattttttgtagagacagttctcgctgtgttgctcaggctggtctggaactcctgggctcaagctatcctcccactttagcgtcccaaagtgctgggattacagtagtgagccactgtgcctggccagtattttatttatttaaattttttatttccgtatgttattggggagcaggtgtcgttaggttacatgagtaagttcttttttttttttttgagatggagtctagctctgtcgcccaggctggagtgcagtggtgtgatctcggctcactgcaagctctgcctcctaggttcacgcagttctcctgcctcagcctcccaagtagctggaactacaggcgcccgccaccatgcctagctaatttttttgtatttttagtagagacagggtttcactgtgttagccaggatggtcttgatctcctggcctcgtgatccacctgcctcagcctcccaaagtgctgggattacaggcgtgagccaccatgcccagccatgagcaaattctttagtggtgatttgtgagattttggtgcacccatcacccgaacggtatacacaacacccagtatgtagtcttttatccctcacccgcttcctaccctttccccctgagtctccaaagtccattgtgtcattcttatgcctttgcatcctcacagcataggtgatcagcttttctttaaaaactttttattatggcaggggtgtatttgacatagtaacttgagttttattaaggcagcattatcagagtaatgctttgggtgacttgtcaagtttcctactcttttcccctgtctaagggtgaaactcttggttggagtaaaggtggtacctgtcctatcattgagaggttatgactaggttatatcatacttggatgaaagatgttttataaactctcccttagtgattgcatgtgatacagaatctttaatggcatttaataatgttaattttaagtttggaatttccaaatttaagattttgaatggaatgggaatataacttgggatttttatctgggatgtactgagaattaaacaactatatcaccttttgtcacaacttagtaaatttttttcttagataaaagttagatttttcagattttattccgttaggatattcatttagagtggaagaactgctgcagacaaaaggaatggtttttagtgtagtgtgttcttagacctgaaaggttttgaaattcctccaggaatctcggatgacattgcctttagcgtgggccaacttgtcacttaaggaaaataattcagtcttttaagaatttttacaaaaagtatagacttgtcttatacttgtacaaatgtatttgatacttttttaaggttaatgaattacattccaaaggatgttgtttgaagtaatgaataattatttgctacccagaataatcaaattttgctgttgtagtacattcttaaactttgttttcttttttttttttttttttttttttgagacagagtctggctctgttgcccaggctggagtacagtggcatgatctcggctcactgcaatgtccgcctccctggttcaagtgatccttgtgtgatccttgtacctcagccacccgagtagctgggattacaggcgtgtgccaccacatacggctaattttatatttttagtagagacagggtttcaccatgttggccaggctggtctctaacccttgacctcaagtgagtcgctcaccttggcctcccaaagtcgtgggattacaagtgtgaaccactgtgcctggccttaaatcctgtgttttgttttgtttgtttttgagacaaagtcttgctctgtcacccaggctggagtgcagtggcatgatctcccctcattgcaacctccgcctcccaggctcaaggaattctcctgactcagcctcctgagtagctgggactaaatgcgcatgccgccgtgcctggctaacttttgtatttttagtagagatggggtttcaccatgttgcccaggctggtctcgaactcctgggctcaggtgatccaccggcctcacctcccaaagtgctaagatgataggcgtgagccattgtgtccggccctggccttaaactttgtttttatgcctctttctaacctaaaccatatttagatgctgaaaatgaatgaaagtgtcagggttaatgcataattctgaagcactttgtgaatcaacagaaaaataattgtttcatagaattttaaatctttgtgtggcatatacaattctgggttgcgtagaatattatttttttcatgcttattgtggaatagagatgcttctgtatttgctcaagaaggacggtggttaaaaaaattgctttattatagtaaaaacataacagtcaattggttttcttgttggtggacccttgctatggcttcaggttcatggttttgaggacctaggagattatatagttaagtctatacactcattaagattgttataatgtgaaagggaataagatctctaaactggggcacatgagcataaaaaaagaaggtgtggaaaatttaaatttataataattgctgcaaaataaaatattacatacaaaaaaattgtagatttacacaatacaattagtaagtaaatggcaaaactcctgtggcaagagtgttcagattactgtcttctgtacattatacacagggacatataacatctctttcctgcatgtgttacacaaataccccatatttcccagtgacccagcttataatggcctcgaggctcctgtgcatcttgcaaggtgaggagtggctgtaacacaactgggaggtacactgagtagccggcaggaatagcaacttcagatatttctcctgtcatcacccaagactagatagagttagctatccttaattgcctgtgtccaagagtacttgattttattagaaaagagtaaggaaggatgtgttaccagtatttggcattcagactgaacgagaagttaaaaatgagaactgtttactatgcttgcaacccacacaacactagacaaagccatagccgaaggtggtattagatataaggtgaaagtttaacaaacccgtcctattttactttaaggcagttattcatagtatgttgttaaaaagaaaaatctgagacaaatgttaagctgggtgggatatcggactaacagatgtacatcaggagagtcctgggcagactggaccatatggtcactccatttatagagcacatattgttctgagtttggctctgatttctccccagtgatgtgtatttaaaatttggctaaaatttaagtctagtgtgtatacattagcattatttttctgagttaattatataataagagtattattctaaaattcttgaaaactgagtgtcattaattacctgtgttttaaaagtggtgtgagccagcagcactgaacctgaccctaaggtaaaataatcttctctagtctgccataacacttgcaagggatgtttcccaaacctttaaagctgcctggacatgtgaagttttctgtttagttactcccgtttgtcactggaaaaacagtaaccttttaatctcaaaactcacagcggtccatgtaaatttaatttctacctcatttgcaatttggagaacgagtctcagtaattcttctgaatcctattatgaatgtaaatttaggactccagatttctaaaattctgctttggacaaattttggatcaacgatctctgattaatttgggaaattttagatgaacaaaagtacctgtagatatatcattattttgatgttattggtagcagtagaacttcattatacttattatttggcaagcaaattaaaagaaattttagtcaacatccatgtcctcaatctggattctaccatttacttttcctaaaaaaatttttataacagctttattgagatataattgacataccatgtactctgcttaaagtatccaaccccatagttcttagtgtattcacagatttgtacaaccatcaccccccatttgtacagtcaaaaaaacattctcatccctcctcccaaaaaaaaacctctacccttcagcaagggtccccattttcctgcaaccaccgctccctcccagtcctagtcaaccacaaattcattttctgtctgtagatttgccaattgtcttagtctgttggggccgctaaaacaaaataccagaaactgagtggcttatgaacaacaaaaatttctcacaattctgaacgctgagaagttcaaaatcaagggacctgcagattcagtgcctggtgagggcccacttcctgggtcatagaagtgcttgatgctgtgtcctcaccttggggccttttttttttttttttttgagacagagtctcgctagtacagtggcgcgatctcagctcactgcaacctccacctcctggattcaagccattcttcttcttcagcctcccgagtagctgggcatgcgccaccatgcccagctaatttttgtatttttagtagcaacggggttttgctatgttgaccaggctggtcatgagctcctgacctcaggtgatccgcctgccttggcctcccaaagtgttagaattacaggcatgagccaccgtgcccagcccctagggcctcttttataagggtactaatcctatccgtgtgggctctgccctcatgctctaaccacctcccaaagacctcacctcttaatattatcacattggtgattaggtttcaacatatgaattttagggggacacaaacattcagaccgtagcacctattttggacattttgtgactagaatcttaaaatatatggtgttttgtggctttcacttagcataatgctttcaaggttcatcttgtagcatgtataagtactttattcctttttattacctgctaatattccattgtatgtataccaccttatatttatccacttatcagttgatgaacattgggttgttttcactctgaatattatgaataatgctgccatggacatttctgtataagtttttatctagacaaaagttttcatgctcttgagtataaacctaggagtggaatttctgggtcatagagtgactccgtttaaccttttagtgaactgtcagactgttttccaaagcagctgcaacattttatatttttgtctgctgtgtggcagtttatgtcaggattctccagaaaaacagtaggctatagatagagaaatagataatagatatatatatagagagatatatgagaggaaatttgttaggggaattggctgacgcgattatggaggctgagaagtcccacgatagactgtctgcaggctggagaacctaccaagctggtagcattacttagtgtaagtccaaagacctgagaatcgggagtccatggtgtaattctcagtgtgagtctgaaggcctgagaaccttggtgggagagggatggggggagggtgatgggagagtggctgctggtgcaagtcctggaatccgaaggccatatcctgaaattcttatatacaagggcagaaggtgtcctagctccagaagggagataatttgactttcccctgccttgactttgttctgccctagcctcagctattggataatgcctgctcacattggatgagggtggatcttcctttctgtgtctactgattcaaatgccagtctctcctggaaacaccctcagagacatacccagaaataatcctttactagctatctgggtattattagtctggtcaagttcgtacctgaaattagccatcacaatttctccacatccttttctgcacttgtctttttttttttttttttttttcctgagacagagtctctgtcgcccaggctggactgcagtggtgctatctcagctcactgcaacctccacctccccagttcaaacgattctcccacctcagcctcccaagtagctgggattacaggcatgcgccaccacactggctaatttttgtacttttagtagagttgaagttttcccacgttggtcaggctggcctcgaactcctgatctcaggtgatctgcctgccttggcctctcaaagtgctgggattacaggcatgagctaccacgcctgacccacttgtctttttgattatagccatcagagtagctgtgaagtgggatctcattttggctatgatttgcatttccctaatgactggttatgtgcattttttcatgtgcttattggccatttctgtattttctttggagaaatgtctgttcaggtcttttttgcccttttttttttttttttttgaggcggagtctcgcactgttccccgggctggagtgcagtggtgtgatctcagctcactgcaacctccacctcctgggttcaagcaattctcctgcctcagcctcctgagtagctgggatttcaggcgcctgccaccatgcccagctaattcttttgcccattttaaaattggattatttttcttttttattgagttttaagagttttttttttttaagtctagagatgtttcttatcaatatgatttgcgattattttctgccattctgtagattatctttttacactttcttaatggtgttctttgaagcaccaaaacctttaattttgaggcttaatttatttcttcttttgtcacttttgtttttggtgtcatctaaaaaactgttggcctcattcaatacatatcacaaagatttgtgtaattacctatgttttcttataaaagttttgtagtttttgctcttatttatgtctttgtttcattttgagataatttttgtatcttttctaaggtgggtttaatctgacttagggttttttaacagactggctcttagatttggatgattgttggccttgttattgaccatggcagagcttgaccatgagtgcccctgaaggctgagggtaacagttgctcattgtggtagtatctttttgtatcagttttgtatctttagtgtcagtttgtggagagaggtggtagtttaaaaaactcactaataggtgttttgttttgcttgtttttattgtggtaaaatgtacatgcagtgaagtgcatagatctttaagtatgtagttgagttttgacatatatatgtactcatgtaaccatcaccccaatcaagatatagaacatttctgtcactcacaacattcccttgaacaataaatagaatcagaagcactctggatttaacaaagttgtttttaaaaaatgcaaacttggtattggtgcagtatttttaagttaaaacttttttcagttattaagaaaatctctgtcggtttgtctgttaggcatattatcttgcctaaggtggtacatcttgggttaaccacgtctggctctctagaggttaatagtgcttctcagtggtttcttttctctgatgtctttccttcattagttaccctcttattaaaaagaaaaaaataagtgtgcgtttgtattttaatgtctaaagccaaagaatcagtgtaatgtttatcataaagacaaagtcaccgagagatgaccataattatagacatttggtacagttttacttacattcttatgtatttctgttagcaaaggcagaatcaaaatatgttattaatatttattgtatatatttatggggtacaatgtgatgttttgacaaagtatgttaataattaagagcctgctatggatgctttgatattagtttttttttttttttttttttttttgagacggagttttgctattgttgcccagggcggaatgcaatggcatgatctcggctcaccacaacctctgcatcccgggttcaagcgattctcctgcttcagcctcccaagtagctgggattacaggcgtgtgccaccatgcccggctaattttgtatttttagtacagacagggtttctccatgttggtcaggctggtctcaaactcccaaccttaggtgatctgccctccttggcttcccaaagtgctggggttacaggcgtgagccactgctcctggcttgatattagttttttaaatgttacttaagctggtggtagaaacatcttcacaaaggcatatgatgatactaatttttaaaattgtactgtggcatttcagttagctattgccacggtaatggtaattaacaatcacaaaatgtaagtggcatacaaacataagcacttattttttgttcgtgagtttgtgggtcagctgacattgctcagctccacttgatcttgtccttttcctgagactagtacactcatgatgttctcatggtggcagaagtctaagaggcaagtagaaacattaagactgcttaaagcagcagggcctggtggctcacacctgcaatcccagcactttgggaggctgaggtgggtatatcacttgagctcaggagtttagaacagcctgggcaacatgaggaaatcctgtctctactaaaaatacagaacattagccaggcatggtggtgcatgcatgggtggtcccagccacctgggagactgagctgggaggattgcttgagcctgggaagcagaggttgcagtgagctgaggtcgcaccaccgcactccaacctgggtgacagagtgagaccctgtctgaaaaaaaataaaaataaatgaagactgattaagacttggtgtactatgacttctgccctcattatattggccaaagcaagtcttgtgcttgagcccagtgacaaaggtgaggaagtatgttctatctataataggacaagctgcagacttaagtgataaaatcatggatacagggaatgatgaagaattagggccaacgatgttatatatggaaaattgaaaacatttgcataagtacagtgaagaatataatgatccctcaggtccctatccagttcagcagttatcaacatgtgaccagtctttcattcatgtttatgtagtgcatcacagtaagagagttacatatgtctggttgtctgtttttgtatgttaagctgcattagttgattcagatgttaccagcttatccgtcattatcaggtttctcattaaccttttacttaatggttttaatagccaccgttcagtgcttatcaaacatctgcaatgaagaccatttaaaaaacatttccagtactgaggactgctacttttgtaaaatacagtaaatttctagacaaatgaaaatttaaaatccaatatattttaaaagcccccaatttttattatagatgcaacagaaataaaattactttgtcaacttgttatgcaaatttatggatgcttatataaaacttatggattctttttttgtcatgaactggtaacagtttgcttaaaaacgttccttcactaactatgtggttaccctggtaaagtcagaatacatgcttgattcattctcattattaatactattttaccaattttgtaaatagttttttcagattttattcattatttccaatggtgcaattaatgtatgctgtataatagaaagggtcgtatgtagaaaagagactgggtttatgaagtacagaaacatcttgaaaaatagaccacatactaccacatactaaatacttgtgaaattgtgacactctattacttgaagtttaattttttttctttttctgtttgagacagagtctcactctgtcgcccaggctggagtgcagtggcactatctcagctcactgcagcttctgcctcctaggttcaagccattctcatgcctcaggctcccgagtaactaaaactgtaggtgtgcgccaccacgcctagctaattttttttgtatttttagtagagatggggtttcaccatgttgcccaggctggtctcaaactcctgagctcaagtgatccactcgcctcagtcttccaaagtgctaggattacaggcatgagccactgtgcctagcctaatttatttcttaaggaaaaaaaatgtataaatgtgttaatattttaggaatagtgatcacgtgataatcaaaattgagagtgttcacaaattcagtatatttagaaaaatacagccctttgattatctttcatctcttcataaaaactgtctcgtattttaaggtcatctttattagtacattgatagtatggggagacaaactgaagagaacgagtcagagtttgggtatccatctgtattgttttcatttcttcgcttctttgcaagatgcctttggttcttcatgcaaacaggctatacatgtgcacatacttaaaatcaccttcaaaagtaatttttgttaaaatactagatctcagtctcctcaggaatgcagagtgagtcaagacaactaaaggtgaatgtaagaaaaaacccaaggagtttaacagaacaactaatcagactgaacatagttcagaacttttccttctaatattttgatagtttgacaacagtttttactgaatgtttacccttttcaaaattgtgtatgttgccagctttaggaaacaagcaggttgattcgggctgattgatactggtttaaacctacgcaaagataaccttgttcctcagttggctaatgcttgttattgaatagaaaaactagctataataaagattttaatctttattatattaataatatattaatattaaaaggatttaaatatcatgaggcaggctgggcatggtggttaacacctgtaatcccagcactttgggaggctgaggcgggtggatcacctgaggtcaggagttcaagaccaacctggccaacatggtgaaaccccgtctctactaaaaatacaaaaattagctgggcatggtggtgcactcctgtaatcccagctacttgggaggttgaggcaggacaatcacttgaacctgggaggcagaggttgcagtgagccaagatcgcaccactgcactccaccctgggcaacagagcgagactctgcctccagaaaaaaaaaaaaaagaaaaaaaaatcacgaggcaaagaacagtatactttcattttagtcaacagtgccaacttttcgtagttatttgagaactggtcacatacgtatattgtgtcaacaaaatagtattgccttaactttctggaaagacaagtcttaggataattgaaactcgaggggaaaattggcgttgttttcaaaggttaaaaaatcagattagaaattgtggccctggaccaactttaaatttcccctcaaattagaagaaaaagataatagagaattgttctattttcttacccctagtgttcataatgtgcagattacatacagtagaactgtattcagtttacctgagaagatagaattcatcaacccaattccatgattagggtatgtgtatggggttctggctgtctttaggtgggtgctagaactggtaactaagttgaagctaaattgctttttagttcattgatttagatagtacattcagctgttcattatattctgggaaggtgcccacatgaacttgaaaggagctattggccccttgtagatagggtagaagagagtctggactactttgaaaatctaagagatgtgtttggttagcaaaagaagatgaagctgcagtcagaggtacaaactggttcagttaaggaggaaaaataattgaatttgaaacaatatacgtaacacgtagaaactaaatagtctttaatgaatacctactagcttgaaatctcattagggaaaagagttttttcaatgtatgaagttattaaccaagagcagtgaaaagctgacataacatatgtcagtagagacttgtatttcccaaatctatcgaacattgaaaacttaaacatagtaatttattgaaggcactcccatagaatcctatctgttgtgtactcagattaaaggaggtagtgaaaatgaactcagatgaattaagtgattgaaatttttgtttatatttaatatctacacatttgttaatataagactaatggaacaaaagttagtgatcataatttaggcaacatgaaacagttggtaattggtaaacattgtttatatgaccaaatgtagatctaatattgtacattattgcatcagggtatgtcttgtgacttccagatttttctcaggtataaacacagttttctaaaggcattttatacatttgttacctaaaatgaacaactgtgtaactgtcagctatacctagaattttttatcagaagtattttagattttggattttggagtatttgtattatacttactgtttgagtgccactaatccaaaaatccaaaatgtttcagatttttggattaaggatgtttaacctgtatatagaaagagacttatttagacaaacttgaaagactcatgtactacttacacatagggttttgttttgttttgttctatgttttgagagaatctcgctctgtcacagcccaggctggagtgcagtggcatgatcttggctcactgcagcttccgcctcatgggttcaagtgattctagtgcctcagcctccggtgtagctgggattacagacatgtgctaccatgcctggctaatttttctatttttagtagagatgaggttttgccacgttgcccaggctggtctcaaactcctggcctcaagtgatcctcccatctcagcctcccaaagtgctgggattacaggtgtgagctatggcgctcagccttcagttggtttttaaatgttttgctgactaaaatattaagggattgcacttgtccattcataattgtaggaataataagttgctacacttttctagggatcttactcaactgttcacagaatacatagagaaatggcaaacttggtgagttatataaaaatctagcatgtctggctgagtgcggtagctcacacctgtaatcccagcactttgggaggccgaggcgggtggatcatgaggtcaggtgtttgagaccagcccagccaacatagtgaaaccccgtctctactaaaaatatgaaaaattagccgggcgtggtggcaggtgcctgtaatcccagctcatcggaaggctgaggcaagagaattgcagtgagctgagaggttgcagtgagctgagatcgcgccactgcactctagcccgggctacagtacaagagtccatctcaaaaaaaaaaaaaaaaaaatctagcatgtctattcctgatgactttttttttttttttttttttttttttgacacagagtcttgctctgtcacccaggctggagtgcagtgatgtgatctccactcactgggtttcatcatgttagccaggttggtctcgatctcctgaccttgtgatccacccgcctcagcctcccaaagtgctgggattacaggcgtgagccaccgtgcccggcctcctgatgacttagctgggctcacctctgtttcttcccttgaagccttgggatgtcttcctgttctatgagaaattccttgagacacactgggaatccctttcccttaaggaaaagcttttctgcaactttatgtgactcaatatgaattaacataatattaacaatggcaatgatagttgtgtcattggactagtggaattttaagatattaatatttaagatatattgtaatttcagagatgcgtggcttctttcacttggcaagtttgaaatgattcatttatgttttggggatttttcagccagaaaggacagcataccactctggagcaatagagaattttaaaagagcagtttggctaaagagtaggccaggttcttataggtcttgggaaatattttgagctttattgaacagtctaaggggaagtcactgtaaggttttgagcaaaggaagatttgaatctgattttttttttctttcttttaagcacactggcatgttatccaaactcagtatcaccagtaatggaacaaaatgacattgtgtctttgcattgtatcatatcaagaggaggcatgataacattaccaatgtaaatttcttgtcaaatgtttatctagaatctgatcatgatacaacaaagagatccagtgtgggacattctacaaaaagctggtctgatctttaaaaaaggtcaatgccataaaagactataaaaaggtgaggaggtgaatagttgaaggagacaatgtatgacaatgtgtgtactgtgtgtgtggtccttgatagattatggaattttttaaaaaccataaacaccttgtttttgacaattggggaaacttatatgtagataattatcaatgttaactttcttggatgtgataatgatattgtagttacataggagattgtccctgtttgtaggagatgcatgctgaaaaggttttgggggagtgaaaggtatacagtttaagtcgttcaggtggaaaaagtgtatgtagatggaaataaagcaaatacagcaaaatgttactgatcattgatatagatgaagagtggtgtttattgtaccaaactttcaacttttctttaggttaaaaaatttaaaaataaaaagggatagggtagaacaacagaactacagagtcttactttttgatttaacattttagtttaggttccaacttcattcagtgttatgtgagatgatggcctcttatgaaaaacctcagtaaaattgaccaacattcctttattttaattattaaaaattttttttaagagatagggtcttgctgcattgcccagactagtctccaattcctgggctcaagtaatcccccctccttagcctaaaaaattatgtgtgcttttaatctaaaatacccaaaggctttttttttttttttttaatccagtaattttgttggagtatgttttagggttggtcgttctgagttcatagactaattttgattcatgttctctcttgccttgtgtaattttaaaagtatcttttagctactatagaaatagtacattctagttttgagctgaggatgtctttcttgcttattctcatagagacatcattctgttctttattccctctattttttatttttatttttttgagacaggatctcactctgtcacccaggctggagtgcagtggcatgattatgactcactgtggcctcaacctcctgggctcctctcacctctgcctcccgagtagctaggaccacaggcacgtgcacccactcccagctaatttttttttttttttttgagatggagtttcactcttattcaggctggggtgcaatggtgtgatctcagctcgctgcaaccttcacctccaaggtttgagcaattctcctgcctcagcctcccaagtagctgggatcacaggcatgcaccaccacacccagataattttgtatttttagtagaaatggggttttaccacgttagccaggctggtctcgaactcctgacctcagatgcttcaccctctttggcctcccaaagtgctgggattacaggtgtgagccacggcacctggcctaatttttgtattttttgtagagacaggatttctttatgttacccaggctggtctcgaactcatgggctcaagcgatctgcctccctccactaccgcctcccaaagtgctggtattacaggctttagtcacagggacctggcctcccttttttttacttttagtaactttgtatgggatttaacttctatactttgtttcctttttatgtgaatttagttttcctcaacttttagaaggaagctagtcaagataacgtttctaacttcatacaacttcctcttctgttaaaaaattatggcacttgtttcctaagaattcctggctctgtccctgtgccccatgttagtctgttccttctccctttttcctgtcctgttagttttggtcatctcccagcagtggctcttcatagcaggccctctcctggaaaggatctctgactggttagtgtcaagggttcatgggggttaaactctcctggcccctttatgtcttaccttgggttccctgccacttgtgtactattggtgtgggcaaacctcctcctagcgcgctgcggttctctaattgggtgctgtgccttctgggtcgtacctcttgggtgttgggaattctcctgttttcaggtcctttaataaggctggattgcttcccacgtaggtgctgtagcactagggtttgtggctgtggtagtttgtcaccaccaacttgtattttggggttcttgtaaatgtctttgctatctagttgttcagtaattatgtgggatccaagaaaatcacaaaacatttccataaacatttccatctccacttttccagaatctgctagatcttcttaccctattcttaaagagtttgctgtatatggtttaattttttccttaatattttttcttgatatcttaaaatatatctatgaatattgcttattttattatgatgcactcagggcagtgagggatattcaccttaagtctttaggcatttgtctttttaatagctttatgtaaatagatatggtagatttttctatcttgtgatgctgaattttatttgtctctgcattgatgtgactaagtttcttttttctcattaggatggtttttctaatatctcgaatctaattatcaaactttacaactgtgtgtgaagtgtgagcaaataaatgccaggttttgagggagaggaactttgcttttactagtgggagcctttttgttttctcctgaattgtcaaagctgtgacagttgctgcataggcaaggtcatacccacagtagaaggaaatagggcaaggattcagtttcttcagtaatctcttgtgtcacagaattgaacatttattgctaaggaaacaggaaaattcttttaaaatacattttttgagtaaagattagtttacttaaataaatacaggcagtccttgctatgcaggataagtaaaacagctaaaaataataatgcaagctgaaaccatgcaaagcaattttaatagtcagtgggaaaattacaattgttttgtgatctttaaaaatttttgttaaatcactaaaaacttttgtcaattacaaaagtgtagggaaataaaaaaatagtaaaactactgtttactacagtataatttaaaatactaggaacattgagaattaaagtattttatttctttgtaaaaagcttatcaatagtagtttaaacagcacttctctgtgtcgtttctgtgccttggttgcttccaacattttatcctttgcagttttaatgtcatgaaatatctcctagagttgctttaatgtgaagtattttcctggagtcacttcctttgggacgttgccattctttccatcacaaccagtttcctcactgatgttggtaagttctgtggctgtatatctagaggctctcaaaccctgtcagccttgccacagtcagcattttcttctgtaactccatatattcaaatttcactccagcattatcacttttcatttctttgctgcattttcatctttgttggccagccccgtttggattatctgtatttgtaaaaagtcctttggggtttatcagtgtaagacaggcaacacactgcactctggctgtttgttgtccattgaccaatcaccaatagaatgaaagaagggaggtgattggtcactgatcctgatgttcatctgttatttacatggtggtttgtggactgaagagctggcaatgaagtttgtcctttatataattactcacagtaatgtatgataactgaaatttgagccatgttgttggggactggtaattaactaaaccttggtataaaccatggtaattgaaatttgtgcatattaggattatgcaaggcaggaactctgcctgtaatgtaaaaaagcatacttagatttggtaaaaatggtaaagataataatgggactgagtctgagaaatttatgcaataatgtagtgtttaaaaatattttttaaaaagttaagattaaggaattatagagaaaatccatgaaggggtgtgtgtatgtgtatacattaatactaagggtaaaggtctgaaaggacaaagttgtagtttgttctgtgatttcacttttctacggtattgaatattcatatgaccagaaccttcatttctatgacctgagtaatttcttctggcaccggattggctccctctcacaaatcctcctctgtagatgactgatttgtttgttactttttttttttttttttttgagatggagttttgctcttgttgcccaggctgaagtgcaatggcgtgatcttggctcacggcaacttccgcctcctgggttcaagtgattctcctgcctcagcctcccgagtagctgggattataggtgcccaccacaatgcctggctaattttgtgtttttagtagaggcagggtttttccatgttggtcaggctggtcttgaactcctgacctcaggtgatccacctgcctcggcctcccaaagtgctgcgattacagctgtgagccaccatgcctgaacgcttgttcgtttttgcagcagacggaatagtggtgttgtaggttaggcctgcatttattttttgcagataaatggagaagggaggaatggtggaaaagattacacaagtggctaaaggactgacttaaggtttttatctgcaaattttacaactagagggagaaaacctccctaaatatgtttattgtaaacatttaggacatattgaagagtataaagaaataaaaatataaaatcacttatagttccattacttagatgaactacttcatcactcttaatattttggtgtatggccagccatactcttccttttttatacatgcatacagacttttgcacttttatataagacacattgaagtggcattatactgtacatgcttcctataagtggatttccagggtacacacaaacttttaaagcgtttctttatgtaaaatttaaaatatacaaacaaacaaaaaagcaaacaaaaaaaacccccaggatagaaaccagacgagtacaatgaacttaatgtacctatcacccaacttcagtgatttcagtacacgggcagtcttgtttcatccatagccacctacttttgcattattttgaagcatcttccaggtaagtattttggtatatttcagttaaaaacttgacattttgccaaattaacctttcagaaaagtgttatccatgtatacctgacagcaatcagatttaaaaagtctttttatttttatttatttttgagacagaattttgctcttgttgcccaggctggagtgcaatggcatgatatcagctcactgcaacctccgcctccggggttcaagcgattctcctgcctcagcctccccagtagctgggattacagtgcccgccaccatgcccaactaattttttgtatttttagtagagacaggatttcaccatgttggccaggctggtcttgaactcgtgacctcaggtaatccacctgccttggcctcccaaagtgctgggattacaggcgtgagccaccgcgcccggcctaaaaggccttttaaaaatcttagtgttatgagttagatgcatatatatcattttcctcagataatacaaaaaaaatcctaacagtgaatttggccatgcttatactctagtgtcaaacttctagtttccccatcagtatatatccctcataaaaatgttgtgcctattgatagggtgaccgaataacttattgtccaaactgggacactttgagattgaaaggatgcactgttaataataatgcccatgtaataaacataaaccaggactatattttggcgcagctggattgtgtgctcatcctaccattagatcatgcatgtaagatatatatacacatgatcactatattacatagtcaataagtggtaagatttattaagtttaagtgtttaatatatgcccatgcatggatatatattaaaatcttccctttaaaaggggtattttgaattggtaactcagaaagtcctaagttttcattcctatgtgtttttggagctgacagtgatcattaatttttttttaagtactgtagctttaattttcataattaagactttgtatttgtattgatatttgcagctggagaatgctggaggagaccttaaggatggccaccaccactatgaaggagctgttgtcattctggatgctggtgctcagtacgggaaagtcatagaccgaagagtgagggaactgttcgtgcagtctgaaattttccccttggaaacaccagcatttgctataaaggaacaaggattccggtagacttttcactaatcttttcatgaggagattgaacttagattgtggaatattttattattaattttcttgagcactgaatttttctacgaaaataagatatggaatggttttatgagaaatttcccatggctctgatttggggagaaaaaggaaaaatttaagctttcagtttaaaaagattaattttaattgtacagttaaaagaacttatcatttgagtattttatgttttataggatttgtgggtcagagttagatgtagctgttgtggccattcattcatttacttaaaagcatttcatcaaaggaaagtaggaagttggctagaatactccaaagagaagaaatctgtgtacaataagtgtttttgcttcctatcccaatagactagtttctaaagtatggagatttcttaaatctaatatatgggctaattttgacagcttgtacccttagaactagtcaaaataacagccagcttatgtagtttcttcctttaacaactatttttggttaatgtcatgctttctttttttttgaggcggagtctcacttggttacccaggctgaagtgcagcggcacgattttggctcactgcaacctccacctcccaggttcaggtgatgctcctgcctcagcctctcgagtagctggaagtacaggcggatgccaccacacctggctaatttttgtatttttagtacagaacgggtttcaccatgttgtccaggctggtctcgaactcctgacttcaggtgatccacctacctaggtctcccatagtgctgggattaccggcgtgagccaccgtgcccggccagtgtcatgctttttaatgcttttttttttattgttgttttttcttagattaagactgttacgtagaagcaagcaactgtagtttatgtccttaattggctcttaaactctaatattagatgtactatgcttctcccaaaatttaaaattttatttttcttaaaagaattgtctttccagtgtgaaagtggttaaaagaacttttaaagacttcacagacatgtgtgcattatcctgtgttttgttggttctgaagtagagtgaattagaaattataaaaggcgggcttaaatttaactctgctctggtgttaggatacagataacaaatgactcttttaaactatggaattttagagtaataagggacccttgagtttatctaattcagctgattttatttaccagcaagaactagttttttagagggatcaaaaaactctgtgataaatctgaatgcctctctgtcaaatcagagctccatcattcccctcacccaaaacaaacaaaaaatccccagcaaactaacagactaaacagatactagaataggatagctgaatacttggcaagagaaatctaatggtgtttcatgtcacttgatcataaacagtgtctaggttcagcttgtacctagaatttcccttatgtagtaggagaatagaaactatactgctgtggcttttattactgtaatcctttctgtagaaataactatttgaccaccccttttctagaacattttgtgacatgagagacatcttaacctacctatttttattttttttttattatttttttgagatggagtctcactctgtcgctcaggctggaatgcagtggtgcgatcttggctcactgcagcctctgcctcgcaggttcaacctggcagattctcctgcctcagcctccggagtagctgggattacaggcatgtgctgccatgcctggttaatttttttatttttaggagagatgaggtttcgccatgttggccagtctggtcttgaactcctgagctaaggtaatctgcccgtttcagcctcccaaagtgctgggattacaggcttgagccacccctcgcggcccacctatttttagaagcttgatctgcctgatacataaaattggtatttggaatgaattacggcttattgggccttgtgattgcaggttactttcagaatagcaggatagaaatggaaggctcaaagagtgattagcttttaaagatttgttgaaataagactttcttaattggtttaagtactacttgagttttatccttaactaagtccatgacttttttgtttttttaatagcactaagaatcttatatcctagagttctttagcagattaaggggtactgggcagtcccatggtggttatcctttgtgaaactaaatataatataatagaatctagaggtcttgattattcagtatgtgttttatagaattttagcttctgttttttttgtttttgtttttgttttttttttttgagacggagtctcgctctgtcacccaggctggagtgcagtggtgtgatctctgctcactgcaagctccgcctcccaggttcacaccattctcctgcctcagcctcctaagtggctgggactacaggtgcccaccaccatgcccggctaattttttgtatttttagtagagacagggttttgccatgttagccaggatggtctcgatctcctgacctcgtgatccacgcacctcggcctcccaaagtgctgggattacaggtgtgagccaccatgcctggcgttagcttctgtatttttaaggagttaggttatagatctacccttaggggtaaccccatccccctttagaattgttatcttaaatatcatttttatcatctgagtagcttgtcttccatctctcttttgcaggacacacactaacaaatgtttaccacatctcaggttgcccatacaaagttcaaaaggtgcttttgagctcattgaagttgctgtttttttaaaaaatgatctagcttgctgagtattttttttttctctcactgttttggtttttatttttttaaattttttgtagacatggggttttgccacattgcccaggctggtctcgagctcctgggctcaagcaatctgcccgtctctgtttcccaaagtgctgtgattacaggtttgagcatccatgcccagccttactgagatttttttttttttttttttttttataaatttgacttatgttgtattgttcttctggatgtattataaatctagcaatatactttgtatatttttatatgacagggaatcactatttaaaaagtgtgttgcagtccaaatttgtaagtaagtttttattagattttacaatgcctttttctttgaaatacagataagtcagatttatctgcaaactcaaagtgagttagagagtaccttaaattgaataaagggcctaagtataccttcattgtgtgagtgggcatgtgtggcaaactggagagcatgtgcagcttgaaggtgcaagccactttatgattccaattggttgtagttatgcaggagtgaaggcccagtgtttccagatttttttttccctgaagaaattccagaaatctgaatttttgtgaaattcttgatattaaaatgtttaagtttttttgagtgaatactcatggccagtgtagatgtattagtggccaggagaggaacataggcctcagtttgcagttttcccttattatagtagtttgactcccaagttaacccatgctctgcaaattataagatagctaaattatactactgacctgatttctgagagcagggcatgtgggggcagaagtgttactagctccctttcctgggctcagggaaaattgttggggatggtggatgctgtaaacaggcaaagttgggtaatgtatactttctattctgccgtcttttgttgtagagtattttgctctgagccatccagggccttcataataccttcagttgttgttctttgacctgctaggctctgagaagacctgtattttcaccatctctagaattcttacgtacttcttccattgataaggaaacaagtaaaacaaatccatgctaagtatcacagatagaaacattggtattatctatttggaagtttgtgcttattaacaagtaaatgctgaattgatagacttctaaacattgcagtaaagtacagtaaaaataattaaaccacgtaggatgacaattcgctgtgatataattagaatttgctacattctaccaaaaacaacgcaatttctggatctgtgttgtttccctcagtggtacaagggagagagggcatagaccttgtataaaaattaacagagattcttcactgattgttccttaaatcacagtgctattatcatctctggaggacctaattctgtgtatgctgaagatgctccctggtttgatccagcaatattcactattggcaagcctgttcttggaatttgctatggtatgcaggtatgtcagcaaatttgttttgaaagcttacttatattttattctgtttgtgagtcatactgtattagtattttctctctttaggatatttaggatacttagttggatgagataacttgtgcatgttacaactatgtgaattgccacactagtatggcagaactggcctgatagggaatctattttcaaagtagtaagtacactgtttcatgtaacaaaacactatccatttaaaaaaaccttggacatgcaaatttacataaaattttcagaaatagtacatagagctcctgtatacctttacccagtactccagttgttgacatttttgaaccatttgaaagttgcacacgtgatgccctgttacttatatttcctaaatattaggacactcttactgcataaccacagtacaactatccaggtcagaaaagttaacatttactcattgttagaatctaatccacagactccttcacatttcacagtttgtctcattaatatcccttataggccacagatcagtcaggactatgtgttgtttttagttgttgtttctttagactctttcagtctgaaatagttcctcagtgtttccttctctttcatgagcttgaaagtatttttaaaaagtacgtgttaattattttgtagaacatttcccaactttggtttctgtgatgattcctcattgttagattcaggttatgaacttttggcaggagtatcacagaattggtgctgtgcttctcagagcatcttatcaggaggcacacggtgtggatttgtggcatgacaggtgatgttaacttggattatttgggtaagatggtatctgtcgggtgtctccattgtaacattaattaataagtatatgtgccttggttttatatcaaaagtaagaataggccaggcgtggtggctcatgtctgtaatcttagcactttgggaggccgaggcgggtggatcacctgaagtcaggagttcgagaccagcctgaccaacatggggaaaccctgtctctattaaaaataacaaaagtttggccaggcgcagtggctcacgcctgtgatcccagcactttgggaggccgaggcgggtggatcacctgaggtcgggagttcgagaccagcctgaccaacatggagaaacccccgtctctactaaaaatacaaaattagctgggcgtggtggcgcatgactgtaatcccagctccttgggaggctgaggcaggagaatcacttaaacctgggaggcggaggttgcggtgagtggagatcatgccgttgcactccagcctcagtgacaagagcaaaactccttctcaaaaaaaaaaagtaagaataagtagggacttactgagatcttgagataagatactcttcttttcttttttttttttcaattactttgtttttagaggggttttaggttcacagcaaaattaagaaggtatagaaatatcttatatacctccaacctctacacatgcatagcttcttccattatcaacatcccacaccagagtgatacatttgatataactgatgaacctacatcaacacatcattattcccaaagtccttagtttagattagtgttcattcttgatttttgtacattctattgttttggacaaatgtataatgacatatatctaccattgtagtattatacattttcgactgcccaatcctttgtgctttacctgttcatcccccatatcctggcaaccactgatatttttttatcgtctttatagttttgcctttttcagcatgtcatgtaggtggaatcatacagtatgtagccttttcagattggcttctttcacttagtaatgtgcatttaaagttcttccatgtcttttcagtctgggtagctaatttctttttagcactgaataatattccattgtatggaggtatcacagttcattcactcattcagtaagtgacagtttacttattcatctactaacgtcttggtgacttccaagttttggtaattatgaataaaggtgctatgaacatccatgtacaggtttttgtgtggacataaatgttcagctttttcagtatattctaaggagagtaattactaagttgttaagagtatgtttagttttgtaagaaactgccaaaactgtgttccaaagtgggtgtacaattttgcattttacagtaaaagcctcttctttcctttcaaagttctttgaaaagtgtttgattatgagtttgacttggcaaaaagaaagatttttttccatctagtagtcttagaagagcctgcaccctgattgagttgaatattggatattggctggcttcttttttttttttgtcccaaggtgtaatatatttatataatttattgattttctttgtcagagtactcagtgttaatcatatagcacaacttatagttatattaaatggttgttagtatattgagaggctttgtaacatttcctaaggaagaatctcacaagatatatttttaaggcctacttatagtgaaatacgtctgaaaaatgctcctttaatttaagagataatggcttagtgtttccccattatgtcagtttcctgatataattataaggccaaattgttctgtcattcactaggatgttttatagccaaagccaggcctgctgatgttaaatgcataacaatttttgcctttattatcagctgaacttttgtagtaagataatgggatatgctgatcttggttatttgggcagaatttgtccttccataaaataatgatatagatgatgccgtctagctaacgtgtgtttgtaaaggactgccacttagtgactaggttgggtaattttaggacctaagtgtatacctttgccccccatgacattctgaaatttgagcaatttcagtgtcttatataccatcttttcccactaactggactctcctgtcagaggctttctaattgcttcctggtgattctttgctttagaagaaaaatgtctatactgctccctttatatctcttaactgatttacatcccccagcctaaaaaatagaatattggtagagcttttgaaactttgtatgtctccattcacattcttctcttcctatcagaggtgaccaactttcctgaattttgttattcattccctttcttttttttatggctttatgcatatgtttatattcctaaataatatattagttttgccgattgacctatatataagtgaagtcatactacctgtattattttcaactttggttttgagattcattcatgttagtacattttattgtaaattattcattttcactattgaatttgtttgaatatagcatgttacttttacattccagtgttcataatgttggctttctagtttctttctattatagacaatattgccatgaatatttttttacttgttttctggtgttatacatacacaaaattttctctagggtaactggccgtaggaatgctgggtcataatgtacattttcagcttcactaggtaaatgatattttttccctcagtttatgttcccacagcaatatataaaaaatcttctgctctatatccctgccaacccttgacatgattagattttttttttttgtattttcttatctgcaaaggtattaaatgggggtcacgttgtagtcttaaagttaattttccttgttattgtaagattcatacgtttatttgccatttgtgtttactatagacacttgttcatttttatattaaattgtctttgttattgtgttgtgggagcctttatatattctggatactaaacttttcccaactatatatattgcagataccttctggattgtgacttgtctcttcactctttatggtttcacttggcttctttatatactcccacaagattttgtcaaattatttttattgggcagaatttccttccctttctcccttcccaatctaaaacttataaaactttgacatataagaaagataagaggagatgatctgattaggtggtagacctggagtactatatggctttctttgaggcagttagaaccttggggtttatccaggaatccatactggccacatactttgctagggcccttgggctgtgaagagacctgaagacatggaggagagaacatagtctattttgataatcactttagtttgaatggactggagtaggaaatgagggggaaggtgaatccaaaaattagacaagggccaagtaaggaagtcttgtgtttaatataaggaacttggattttatcctgtagaccaggcatcagtaagctatggcctatagaccaaatctggcctaccacctgtttttgcatgacctgcaagcaaagaatgattttttacattgttaaatgattgggaaattgaataaaaattatattttgtgatgtgtgaaaaataaaattcaagtctgtgtccatgataaggttttattgcaacagtcacacccatttctttgtaagtattgtctatggatgcttttgagctgcagtggcaagagctaaatagttgctacagacacagtagggcctgcagaggccaatatttttctctggccctttaagaaaatttaccaacccttgctgtagatgctagagggctaatgaagaatttaatcatagactaacacaatcatctttatattgtagtagctaacattggtaggtctctagaatattgctggaagatgggcaagactagagtcaagggaaatgattagactgtagcaatgtttcttaaattgcatattagttaatgtgctacagaagagagttttgtttttgagtctttagtttgacaaatactgtgttaaacaagtgctagcaggtgtctttactgctggagtgagttcaaacctttattgtgggacagtttactgtgaatgcctaagaggagaatgtagagggcaccatttgctaatggttttgaagaatatgctactctgagttataagaatgaacatttatttaagtcatctgtcttattttctgtcaggggatgggtcaacctgcattacacattgttaaggcagatatcttctgatatctgttgtggaacagatgcactaagatgagtgggagtgtttatatcactgcagatctaatgtttgcaaaaatcttctgtatatttatctgatatgggaagtttggagcatgtaaatgataaataagaaaagaagattgattaacagtggaggtgctgaattgaagagaccatttgaaatcagtcgataagcgtatgggacttgaacagatgagcttaactgaaaggccagattttcttgcttatcccatctctatagcacagccttcggcaggctttcagactgttcctttggagccaaagactaaatagcttggaaaaatgtattgtctaattcttgttctggtacaaggttcttgttgtctgctgtatctttttctaaactcagaataccttttaaaatgcttttaaatcaaactcaggtgtaagtaattaagatatctctatttttaagcagatgaattcattaaagtacactaagtggaaaatctgccagtgtttgacagaaattaactgcaggtcaaattttgaatccaattttgttttataacaatgcctgttaataactgtatagttatatataacaataccaaatggtctacatttattaaactccttttattttggggatacaggtatatcaggtattctgaaaagaaataatataaattattaaatcagtataacaaaatgggtatttttcttttgatttctattaacattaatttttttctttgaacagatgatgaataaggtatttggaggtactgtgcacaaaaaaagtgtcagagaagatggagttttcaacattagtgtggataatacatgttcattattcaggtattacatttttagatgaataagaacaatagtaattaactaatttaaagttgatactattatttttaacaattggagattctataattcataaacaaaattaagggtatagtagtgaccacaagtgtaattgtcatatttgccttccagttgagtgtcaagacagtgtgggttggtggtgatgttaatagggaataaggaaggtatttaagaaatttgaaaacgcctgtgcagttgtggaaccctacttggaatcgtatattgtgataccaacatttgtattttcccttttcactctttgatctgtttttaaagattattatttttctttttcttttttttttttcgagacggagtctccctctgccacctgggctggagtgcagtggcaccatctcagctcactgcaacgtccgcctcctgggttcaagcgattcttctgcctcagcctcccgagtagctgggactacaggcatgaaccaccacgcccggctaatttttgtatttttagtagagacagggtgtcactatattggccagattagtctcaaactcctagacctcgtgatctgtccacctcggcctcccaaagtgctgggattacaggtgtgagccactgcaccctgccgaagattattatttttcaaaggaagattaataaaaaagccaaggttgctcaaaataattttttaaactgaagttactcattgtcaaaatgagtttgaaatccagtgcttgttcagcttcataatggagaggtgttgacacttacattcatccttatttcatgtgactcatcttgagaaacatttcaggaatcttgtatgtgatataccagaagaagctttaaacataaagatttggttaagatttgacatttttctgtctttctagttctaaaattctgggactctatcacaacctatactattcctaaacaacaatgtcaacaacaaaccccaaaatatctttttgacattaatgtactttatcttcttgctttatttttaatttacatttttattccttagtattatttattatttattttgtaatttaatttaattttttttttgagacggagtctcactctgtcgcacgcccaggctggagtgcagtggcaagatctcgactcactgcaacctccacctcccgggttcaagtgattctcccacctcagtctcccaagtaagtgggattacaggcaaaattttttgtgtttttagtagagacggagtttcaccatattggccaggctggtcttgaactcccgatctcaggtgatctgcctcggcctccgaaagtgctgggattacaggcatgagccaccgcgcctggccgttccttattattatttaatatcgagttcttagtcagattttccataacgtaaacagtatatttttatggttgatttgtttgaatcagggtccagagaaagtgtacacattacatttgattaatgttacttaagtctcttaagtaacacttacttaactaagtttcattaatttgtttggcactagaggtccacattggtactgggttgtaattgtttctaggactttcagataacagagttgggcagtacctagtcttttagaattgggaaacagaatcattagtgtatacttatttccagttaaaatgaaagattatagggtttttacctaactcttttgattttgtatctccctactcttaagctgaaaatgttgattactaattatttgttttaacctacagtatgcctgtatctatattgtacttacagttacaggataacagtttcagtactaagtcctattactacttacataagactaccgaatgaagtgttaaggtctttgaaatacctctgttcttaacataaatctcaacagatatataatgttgaaattattgttctgtaatctcttgaaattattcctgtgtgtctggttatactaccaacttcatatatttatttgttctagtttccttccaatttttatggattgccttttttaaggtctaaggttttttattggctttatttgtaatggacataatatttgaattctagcagtattccattgtcactttttttttttctgtttttctaatcatgtatggcaagcttgtgtttctaaaacaaaagaacccatgaatcatgtttttaattaagatatttgtgtgttttattttttgtatgtttaggggccttcagaaggaagaagttgttttgcttacacatggagatagtgtagacaaagtagctgatggattcaaggttgtggcacgttctggaaacatagtagcaggtgaaaattctaaaaattttgcagagttcatttaaaaaactttatctgaagagtaagcatatgcttctgtatgaggtactctttgatttttcctttttcttctgatttttcttctcttttttttaaagataataatgaatgtaggattaactgttatgaagggaatttaataataggaggattatccagaagtctctcacttactggtttactggtttgctttgaaatacgttgaaaaatgttcatcctaatataagcttttcaaacataaaacactatacttttgtaactcaggaaaaatttgagaactcgctgtcttcatactatactatattcagagttacttgtcttttattcctttcttaggtgtacatagcccatagcacccaaataagtactcaagacatttccctgaaccagggttggcaaactgtgacttgtgggtcaaatctagtcttttgcctttttttaatgtccaggagctaagaatgatttttacattattaaagggttataataaaatacagggactgtgtatgttgcacagagcccaaactatttactctctagtcctttgcagaaagtttgctgacccatgctcttaaaccattttgttctctaattttttgaggaaggggaattgactttctggctgtttggggttttgacttgcttacctgaataatgtaaatggtcactaatattcctggaagttagtaaaagaaaaaggtgggtttatcactaaagttctctttgagataccacacctaaataataagttgaaaaaaagatttgtcatggacatgctgaatttagtaatctgcctgctgttacttaacagacattagcataatgtatgctaatatgttagcataggaatgcccaagaatggttttatactaaaagttttttacttcataaaaaaagatcaacagtggtagataagcttgtgtatttttattcatccaacaaacattcgagtacctattatgtgtcatgcacagttctaggtgtcaaggatttagcaatgaataaaataggccaggtgcggtggctctcacctataattccagcaccttgggagactggaaagtggatagcttgaggccaggagttcaatccagcctgggcaccatagtgagaccccatctctacataaaaataattttaaaaaaattagccaggtgtggtgatgcacatttgcagtcctagctactcaggaggctgaggtgggagaatcatttggccctaagagatcatgactgcggtgagctgtggttgtgcccctacactccatcctgggcaacagagcaagaccctctctcctaagaagaaaacaaaacccaaaaccaatatagcaaaaatgcttgtttaacactggaaaaagggaaagtaaacaataaagaaatacagaatatgtcagaatcagatagtgtttacattgaaaaaatgggggaagagggtattctggaatatgtgtgattagggttgttgcaattttataggctattcagggaaggcattcctataagatgacattggacatgaagaaaataagtgctaacctttttggatatttggggaaagaggaaacagcagtacagagatcctgggtggggaccatatttgaggagcagcaggaatccatgtggttggagcagagtaagcatggtggggagagtgaaagaaggtgaaattagagaggtagttggatgccagaacacaggacctttgggccactgcaatactttggctttactcatagtgaggtggggttttaagtgaaggagagatgtgatctaatttaaggattaagctgtgatgaggagaaaagatacagggtgaaagagaagcaagaagacaagctaggaatttattgtagtaatcaggagagagacagcgttgtcaggatcaggatagtagtgatagatgcaggaggaagataaaggggagggtacctggagaatctcccaccagcctgcctactgggagaacagggtggggccacaggaagttctcaccctttgcaggagggaggagcctggcctttcctgttcctgtgtggtgacctcggattcaatctgtgagatgggggcctgttaacaggaagccttctcactttgctgagagtttttttccctttttctcaataaattccattcccccttacccttcaaagtgtctgcgtgcctaacttttcctggtcatgtgaaaagaacccggttttttctacagcagtagaagaggaagttgttaggaccagggtggcagagatagaagttgtcaggactagggtgatagaggtagacattgttaggagtgattggattccactgttttgaaggcagagacagaggatttgctgttagattgaatatggagtggggaagaaaaagtagaatcaagttttagctcccaagtttttttgcctgaggaaccaaaaggaagatgtttctatttacctgattggaaagacctcaggagtttgagtttgcatattaaattcaagatggtttattaaacatcaaaacaaaggtgctgagtaggcagttgtatatatgagcctggactttagaggagaaaactgggctggaggtataaatttgggggtttaccagcatacagacagaattttaaggtgatgagagtatgagatctcttagaatatttagaaaagagatttaagggctgaggactgagtaacttccatgtgtagaggtgggcaagatgaagaggaaccagcaaagagattcagaaggaatagacagtaaaataagtgaaccaagagagtggtatgctgagagccaaataaagtgtgaagtactgtggtatagtagaatgagtatgagtttgggagcctgtcaggcctgatttagttacttaattacctgagagttcatacaagtttcttgatatctcagagccataaaataggagtaacacatgcacatgtaaaatacctagcctaacatctattgccccataggtaatatttgccagaaacacatttgtcaagtagttgaatatatttttgtggttaatagtagtcaagaaagaaacagtatattctgacaagtctaatatatgagattcttttaatataagaacttggtagctgtacaatgcatttgaaagccatgtgcaactgagaaccaaaattgaattatcaatgcactagatagacaaggcaactgaagtaacttgggtggagaggtgagaatactgtgaaacaatctctatgtgggacttaatgtacgtcctatacctcattaattgctgtgtcaagaaagacctttataaagaaaggttatagcaatgtagtttttgtctttgttatgtatttcctattcctatagctaaaaatctactgagacataccagttttaacatttatagaggaagaaggctgggtgcagtggcgcacacctgtaatcccagcactttgggaggccgaggcaggaggattacttgagcccaggagttcaacaggcaacaagataaaaccccatctcttcaaaatatttaaaaattagctgagtgtggtggcatgcacctgttgttccagctactcaggaggctgaggtggaaggatcacttgagcccaggagatcgagactatagtgagctatgattacacctctgcactccagcctgggtgacagagcagtaaataccctgtctaaaaaataaaataaaataggccgggcgcagtggctcacgcctgtaatcccagcactttgggaggctgagatgggcggattacaaggtcaggagatcaagaccagcctggccaatatggtgaaaccctgtctctactaaaaatataaaagaaataagccgagcgtggtggcacacacctgttgttccagctactcgggaggctgaggcagaagaatagtttggacatggaaggcagaggtgacaagtgagccaagatcgcaccgctgcactccagcctgggcaacagagcgagactccgtctcaaaacaaaacaaaacaaaaaacaaacaaacaaataaaattggctgggagcagtggctcatgcctgtaatcctagcactttgggaggctgaggcaggtggatcacttgaggccaggagtttgaggccaccaggccaacatagtgaaaccctatctctactagaaatacaaaaaatcagctgggcgtggtggcacacgcctgtaatgccagctacttgggagcctgaggcatgagaattgcttgaacccaggaggcagaggttgcagtgagccgagatcatgccactgcactccagcctgagcgacagagcaagactctgtctcaaaaaaaaaaaaaaaaaaaaaatgaaaccatagaggcagagattgtgagaaattaattgtggttattgcttgagtcttttcagcatgaaaaaattttaatttgtgactattttctctataaaggcatagcaaatgaatctaaaaagttatatggagcacagttccaccctgaagttggccttacagaaaatggaaaagtaatactgaagaatttcctttatgatatagctggatgcagtggaaccttcaccgtgcagaacagagaacttgagtgtattcgagagatcaaagagagagtaggcacgtcaaaagttttggtaagcaaattattatctggaagtctacaaatttatatcaataatattggaatccaggagttagagtcctcaacacagcccttaaatgttctacagttttagagtgcttttccagttaccatacatatctttggtataaatagcacttttagataaagatttaagccaatagtgtttattttctttttgcttgttttgctcattttgatttttcattttaccccgtaggttttactcagtggtggagtagactcaacagtttgtacagctttgctaaatcgtgctttgaaccaagaacaagtcattgctgtgcacattgataatggctttatgagaaaacgagaaagccagtctgttgaagaggccctcaaaaagcttggaattcaggtcaaaggtattgaagaacctcagaaaagttaacttacatgttaggacttgtttaatcaaatctagtcttatggtttaaatgagcacaattacagttttctcagtatgctcaaggttgaaaatgttttttatttttacaattacctctggtgtaagagaggtctcagtataaatttttaaactattgattctgctaatttaatacatagattgataaattaacattttctagctactgttcagttctcttctagtcattcacttttaaaattatcttttcctggccaggcgcggtggctcacgcctataatcccagcacttagggagatcaaggtgggtggatcacctgaggtcaggagttttgagatcagcctggccaacatgatgaaaccccatctctactaaaaatacaaaaaattagccagtcttggtggtgggcgcctgtgatcccagctactcgagaggctgaggcaggagaatcacttgaacctgggaggtggaggctgcagtaagcagagatcatgccactgcactccagcctgggcaacaagagcgaaactccatctcaaaaaaaaaaaaaaaagaaattatcttttcccactctatttaatgatatttatgtacgaggctacccaagccctcctcaggtatgggtatacagattattttgcaaacagaaaagcttattgtgggagaggtaaagattctatactttttttttaaagggccactagtaaacttcttagtccttaataacatttacttttgtagataccttttatactttttaaaaacaaggtgcaattgatagttataatgaaatgaggtcagtagtttaatgataaaagttattgtacatattatggaattaattggaattgataccaaaaggctgaataaaatgaagacttgtaggtaagtatgatgtgaacatgagtctatttttttgaaacttctctaaaataacagtaaaagcataaaacagtgtaataccttaaggatgtgggatagaggagatgacagaagacagatggtttcaataaagtgttggaaagtggaaagcagatgggtaaaggtggattttctctgtttaagctaggggggtctctcccaacatcctgggaaggtccccaatggtatcttcacatggtgataggtttttgtgaaatttgcaagatattttgataatttgtagttgtttattttctttcaactctgacttgttttctgttaggtggtgctggaatagctgtgggcattttgaaatcaggctaagacaaagatgagctggggatgtgcttagtatgggtttagtggtatatatgtgatttgcaattatttctgtgtatagttaagtacttgttagccatcccagtataacttaagatttttagtatcttatttgttattctggttcttgaaggagtccttcaaattgtaaaacttggatctgtccctggtaactgacatagcagaatggaggaattcacaaccttggggtggggtgggaaggaagccaataaaaagcaagctgatttgtgctgtagacttctggggaggctcaggaatttgaagcattgggttctttttgggacattggtctctgggttagcctaaaacagggattagttaaaaatctgagaagcagatccttttccttagcacaggagcaaactgtgctctccttccccatcagaacactgtggatgtattctctagaggggctaaacgagaggttctgggcttgagcctactaataccaggcatagttgagggtggaggtaagactctacattaagaacaggattaagtaaaattctctatgctgaatggtgattccctcttcctaccacttcctctgtttaactgccagaatgccaataggcaggagagtagatatgtcttctctgggtagttgaccaggtacaggaaagacctacaaattcactggcttttccacaatgaaatagccagctgccttgtcatcttacggtgaaggcctccagtagctaagttttattagcaaatagagatcttagtttttagtttcttgtcattaaataaaaagccaaggattgttaaatgtatgttcataaatattagttttacaagacactagggtgtgattccaacagctttgactccgtttaggagccggtgcttttgacttctttggggctaagcttcttgctgtggcttctgcttgttgctctggtgaagcactgaagagagggaaaacaatcagcataaattttttttttttttttgagacagagtcttgttctgttgcccagggcagaatgcagtggcacaacctcggctcactgcaacctctgcctcccaggtttaagcaattctcttgcctcagcctctgagtagctgggattgcagacatgtgccactacgccccactaatttttgtgtttttagtagagatgtggttttgccattttggccaggctggtctcacactgctgacctcaagtgatccacccaacttggcctcccaaagtgctggtattacaggtgtgagccaccgtgccggccctaatcagcataatttgtgatgtcatggccctagatctaaatgccaggagattctcacagaaacaaaatttaatgagtgcctgtatctgaattctgttactcaggatttattgatagttctttttcttttcttattttttttttggggatggagtctctctctgtcacccaggctggagtgcagtggcacaatctctgctcactgcaagctccgcctcccgggttcacaccattctcctgcctcggcttcccaagtagctgggactacaggcacccgtcaccacacccagctaattttttgtatttttagtagtgatgggatttcaccatgttagccaggatggtcttgatctcctgacctcgtgatccacctgtctcggcctcccaaagtgctgggattataggcatgagccaccgggcccggccaataattcttgaatattgatagtttattgaaatttgtaggcagatttaggggagatcaaaggactgctcttcagactgggaaagaaatagcttttctttctgtattttgacttgttatttttgattaaaaaacatgttataccaatttaaaacgcttctcccctttcctccagtgataaatgctgctcattctttctacaatggaacaacaaccctaccaatatcagatgaagatagaaccccacggaaaagaattagcaaaacgttaaatatgaccacaagtcctgaagagaaaagaaaaatcattggggatacttttgttaaggtacctttgtttttaatatcctcaacatgtactatttttgatgtgaatcttagtatttgtttttccttgtcactatatcaaataaccaaattagctaattttaatcctttttcttatgtggctgagagttttattgtatattgtcattatattgaccagtagaaactgttttatgtttttttgttgttgtttggttttttttttgtttttttgagacagagtcttgctgtgtcaccaggctgggctggagtgcagttgcacgatctcggctcgctgcaacctctgccttctgggttcaagtgattctcctgcctcagcctcctaagtagctgggactacaggggtgcaccatcatgcccagctaatttttgtacttttagtagagacgggggtttcaccatgttggccaggttggtccggatctcctgacctcatgatccgcccgcctcagcctcccgaacttctgggattacaggtgtgagccactatgcccagccaactgttttatgttttaataaacatgtttgcctacattgccaggctattattttaagaatagatgtctttaagagtagatgtattcagcttatcatagactttaattcattgattatctcaatggctaatttcaagctcttgaatggaatgttcacatacctttgtttttgtttttttttttttttggaggcggagttttgttcttgtcactcaggctggagtacaatggagcgatcttggctcacggcaactttcacctgctgggttcaagcgattctcctgcctcagcctcccgagtagctgggattacaggcatgcaccaccatgcccagctaatttttttctttttttgagatggagtctcactctgccgcccaggctggagtgtagtggtgcaatcttggctcactgcaaactttgcctccagggttcaagcgattctcctgcctcaacttcctgagtagctgggattacaggcgtgtgccaccatgcccggctagtttttgtatttttagtagagacggggtttcaccatgttagtcaggctggtctcaaactcctgaccttgtgatctgcctgccttgcctcccaaagtgctgggattacaggcgtgagccaccacacctggccactaattttgtatatttagtagagatggtgtttcatcatgttggccaggctggtctcgaactcctggcctcaagtgatccaccccactcagcctcccaaagtgctgggattacaggcgtgagccactgtgtttggcctcatgtacctttttgaatcgtgttagaggacatgttcattaaggagtttattttggagattggtggattttattttctgatcagtatttctaaaggactctaagagatttagctgaaagtataaacttttgcttttaaaaaaagttatctcttacaaggctgttttactcctgttgattatagattgccaatgaagtaattggagaaatgaacttgaaaccagaggaggttttccttgcccaaggtactttacggcctgatctaattgaaagtgcatcccttgttgcaagtggcaaagctgaactcatcaaaacccatcacaatgacacagagctcatcagaaagttgagagaggaggtaaaagtttatgaaaactttttcataaagtagatacatggaaagtcttccattcttccctcctcttccctctaatattcacagaattgcataaccatcaccacatctaattttagaacattttccttaccctaaaaaggaaccttatacccattagcagtcactcctcgttctacctgttgttgtcattgtcatcctgcccccaccccccagctcctcctttcctcctttcttgccagccctaggcaattaccaaccactaatttattttctgtctctatagatttgcttattttgggcattttatataaatggaatggtacaatatgtggtcttttatgactggcttcttttacttaccgtaatgttttcaaggttcatccatgttgctgcatgtatcagaactttattcctatttattgccagataatattccatcttatgggtataatgcattgtatttatcctttgatcagctgatgggcttttgggttatttccactttttggctgttataagtaatgccgccatgaatactggtcataagtttttgtggatatgtgtttttatttcttttggtatatgcctgggagtagaattgctaggtcttaaggtttaacattttgatgaattgccaaactattatttaaaatgatgatacaattttacattcgcactagcaatctatgagattcaaatttttccacatcctcaccaagacttgttgttatctcttttcttattgtaaaaaacacataacataaagcttaccatcttaactctgatttttttttttttttgagacagagtctcactctattgcccaagctggagagcagtcatgcgacctcggctcactgcaacctctgcctcccggttcaagagattctgctgcctcagcctctcaagtagctgggattacaggcatgcgccacgaggcccggctaattttgtatttttagtagagatggggtttcactatgttggccaggctggtcttcaactcctgacctcaggtgatccgcctgcctctgcctcccaaagtgctgggattacaggtgtgagccactgcatccagcctcatcttaactatttttaagtatatagttcagtagtgttaggtgtattcacattgttgtaaaacagatcttcagaacttttcatcttgcaaaacaaactctatacccgttaaaaaacaactctcccttttttctggcccccaccctctggtaaccaccattctgctttctgtttctatgtttcactactttaaatacctcatataggtggaatcatacagtattgcctttttgtgacttgcttatttcacttagcataatgtccttaaggttcattcatgttgtagcatgtgacagaatttccttcttttttaagactgaaaaatgggccaggcacagtggctcatgcctagcgctttgggaggctgaggcgggtggatcacctggggtcgggagtttgagaccagcctggtcaacatgatgaaaccccgtctttactaagaatacaaaaattagccaggcgtgatggcgggcatctataatgccagctccttgggaggctgaggcaggagaatggcttgaaccctggaggcggaggttgcaatgagccaagactgccattgcactctagcctgggcaacaagagtgaaactctgtctcaaaaaaacaaacaaacaaacaaaacagactgagtaatgtactactatatgtatataacacattttgcctatccatttttgtttatcttttggatatatgcccagaattgggattgctggatcatatggtagttctctttttaatttttttgaggaacttattgtccatagcaattgcaccattttacaatctaaccaaaagtacataatgactccaatttttccaaatgcttacaatttgttattttctatttttttatggttgtgttatctgacttttaaaaattttagtcatctggctcagcacagtggctcatgcctataatcccagcactttgggaggctgaggcaggtggatcacttgaagccaggaatttgagaccagcctggccaacatggtgaaaccctgtatctactaaaaatacaaaaaaattagccaggtttggtggtgcatgcctgtagtcccagctactctggaggctgaggcaggagaattgcttgaacctgggaggcagaggttgcagtgagccaagattatgccactgcactcccgcctgagcgacagagcgagactctgtcttaaaaaaaagtaaataaaaatgttagtcatcctagtgtatatgaagtggtatatctcattttgatttgcatttccctaatagctaatgatgttgagcattttttcatgttcttgcttattggccatttgtgtatcttggagaaatgtctattcgtatcctttgctcctttttttaaaaaataattttaagttcgaggatataagtgtaggtttgttacataggtaaacttgtgtcataggggtttgttatacagattatttcatcacccaggtattaggcctagtacccattagctttgcttattttttaattgacttatttatttttttattattgagttgtaaatgttctttatatattctggttacaagtcttttatcagatatacgacttgcaaatgtcttctctagttctgtgagttgtcttcattttctcaatgataacatttggagaattataattttggaacactagtatattttctaattgctttgtattctaatgagaattagatcagagatgatggattggtttaccctgatttatttatatttaaaaagcttatgtttaaaatgtgcttcaaagagaaataatacccataagatattcgtctaattccttataggcacttaggaacaaatactttaaaagatgcatttttagtaataatctgttagtctaagaaaagtggtaacaggaaaagccaataatttattacgctttgttttccatgtcatcttggttactagtttatattggttggcttctttcctccctgtagggaaaagtaatagaacctctgaaagattttcataaagatgaagtgagaattttgggcagagaacttggacttccagaagagttagtttccaggcatccatttccaggtaaaaattagaactgaattttgtttgattcatctttagaccttcatgttgaagaaaaatcaattcagacaattctgaaataatctgtcatctcagggaatatgtaacatgagagaaaggaaaggatggttagggaataattgaaatcttttgagtatctactgtatttacttcattttattaaatactcattttccattgtatctgcatttaagtgttacattatccttgggagtctggcatctgatgttgccatcagatgagaaaaccaagacataaagatattttaataatattgccacacagtcctagtagtagagctaggatttgttatattatttgtaccataccgcagtgcgttccatggaagatgtgaggatttaaatttagctctttaaaatccttgtcctatgtctgacttgtttgaatggatgaaccacttattctgtgcagagaattcctggcacaatgtaactaattcctgaacaaataatacttcatttgctgtcatatagaaaaaaattagaggtgatacttgtttaaagttaggactgcaagtcttaacctgtttttgttactagttcttgagaggttgggcaagtctgtacatcaggttctagctcactgtaatagtgattaggggatgatttgggagaatgacctaattgaattgaaaagcataataatagccaacatttcttaagtactttctatgtgctaggcactctgctaaatacattttattgtcttatttaatcttcacagtggaaactactattatcctgttttacagatgaagaaacttagggacagcgactaatttgccaaaagttatgcagattgaaaggagtggagccagaatgatatccccaataatttttatctcagagtttatactcttaatgatcatattggctccataaacagctttacacatgtataacctatgtggtgattagcacttgttctcaaatcaagtaatcagattgagatctttagtccctaacctgcatgccggcgaagaatggtctgtggaccggtgtggtggctcatgcctgtaaccctgagtctactaaaaatagaaaaattagccaggtgtggtggcatgtgcctgtaattctagctactcgggaggctgaggcaggagaatcgcttgaacccgggaggcagaggttgcagtgagacaagattacaccacagcactccaacctgggcaacagagcatgactccatcttaaaaaaaaaaaaaaagaaaaaaaaaaaagaaaagaatgatctgtgaatgtaatggagggaagccaagtgttctatgtccttggtcattatttcatctagactcacttgatgttttaaaaatctattttgtatgttgagttttctattatgtaagacttcattggcaaaaatggttttactgctttaaaaactatcttaagtatgctgtttattctaaaggtaaagaatggactagggaattaattggacatagctgaggttttgcctataactaggcatctcaagtgatgccttttctgttgtagtaaagcagcaggatccagttttccatagatctatgcacaccttgtgtttttatcaacttttaagtatttggtaaaatagaggttgggtacagtggctcatgcctgtaattccagcactttgggaggcctaggcaggcagatcacttgaggtcaggagttccagaccagcctggccaacgcggtgaaacccgatctctactaaaaatacaaaaaattagctgggcatggtggtgcatgcctgtactaggtacactggtggctgctaggggagggtaaggcaggagaatctcttgaacctgggaggtggaagttgcagtgagctgagatcgtgccactgccctccagcctgggtgacagaatgagagtctgtcaaaataaaatagaaataccagttcacattttttggtattaaaaactcacattatttattagattatgtgtatattttaacagtataataccattcctaaaattagattgatagttgaagctggatgataagtaaggtagttaattatactgtcctctttatgtatatgttggaaaattcccttattaaaaactttggatgagcacatatgttataaactaaaaatatttactggataattttgtgatacttaatacatggactgtaaaaataggataggccaagtgcagtcagtggctcatgcctgtaatttcagcatttccagaagccgagcttctggagttcaagaccagcctgaacagcatagtgaaacccctatctttactgaaaaaattagccaggcatggctgccggtgcttgtaatccaagctatttaggaggctgaggtgggaagactgcttgagctcaggaatttgaggctgcagtgagcaataattgcaccactgcactctagccttggtgacagagcaagactctatctcttaaaaaaagggagataggaatgatagggagagaattaaacatactgtcttatttgagcctcacaatggaaactatgatagtattaccctgttttatagatgaagaaacttagggacagagattaatatgccaaaaagttatgcagactaaaacgtatgcataatttaaatgttgcagcattctaaatgttacaacatttttagaatgttgaagcattctaaaaaatgctgagttgaatgggaaactaatagctacatttggatacttatgatgggcagatggattttaattttggaaaggttttatgttttgaaactagtggcatttttatattagatttttatattagaaatataacattaatgttaaatactattattactcctacctttaggtcctggcctggcaatcagagtaatatgtgctgaagaaccttatatttgtaaggactttcctgaaaccaacaatattttgaaaatagtagctgatttttctgcaagtgttaaaaaggtaatatttgatacagctaatcattacaagaaattgaacggattcttattatataccagcatttataatgaattttttagggttttattatttgaaaatttggtgtaatataacctttgaaatgactgtggagaaaatattccacagatattcttatatgcatatttttctttccagccaaacaagactggatatcctgagtctctaaaaatgtcagataaagtattttaaagcactcttttatgtgtatttcaagatagtaagggaaatcctttgagtcaggaatttattgggagcatcatttcaagaaaccaaagctgtctgctcctctgaggacagctactaatcctttgtggtctaggatttgggtttttacaatttcattcatgtacaggtgtctgagtatgagactgggtccaagaagggccagagattggatcagatatcttcacactatgcaaccctttagctccaaattgaagatgaatggaaaaattccccagtctactttattctacagatttggggttttaatttatgtaatgtgtgtggtctgaaaattgcaacgaaataattttaaagtggatctgggttggtagtgcttatgggagttaggcaaggaaaaatgcagattctctttagaatatcttcacctaggtcccaaaggattctcatagatagatttccaacaaatatgaggttataataaaaaatacaaatcacatatagaagtatggcaccatgaatgagaaaggaaaaaactgtcagaacaagaccctcaagactttactggaattaacaagcaatatgtaaagtaaatagaaataagctattcataataagaataatgtataagagactactaaaaataactgggcagatttgaaaataatctaagttctgggaatgaaaataataactgaaaaacagctgaaagagagaattaatgaactaaaagaaagttgtttagagattatccagaaattaggacaaatcatcataaagaaaatatgggtagaaaaggttaagatggaaggataaggcaagtgctaacatatgtccagaaggaaataatagaaaaaaatgtattaattcctccacactggtaaaagacatgatggctcagattcaggaaatgtaacacatctcaagcagaataaagggaaagaatttgacacctagtaagcacatcttagagaaattaaagactgccaaagacagagcagctgcggagaacagatcaattacctacccaggaaattatactgaaacagtaaaggcaagacttcaaaataccaagagaaaataattaactgtagtgaacagctaaactgtcttttaaaaacaagggcaaaataaagttatttcgagattaaatagaatttactaccaacagtcccttactgaagaaactaaaggcaatgattttcaaccctggctgtatgttagaatcatctggggaagcttttgaagtatactattcttgacatatagccctagaatttaattggtctgagatgggacctgggcattccaagtaatttgtgcagttaaggttgagaaccattgtttttttttttgtttgtttgttttttgagatggagttttgctcttgttgcccaggctggagtgcaatggcacgatcttggctcactgcaacctccgcctcccaggtttaagcgattctcctgcctcagcctcccgagtagctgggattacaggcatgtgccatcacgcttggctaattttgtatttttagttgagacggggtttctccatgttggtcaggctagtctcaaactcccgacctcaggtgatcacccgcctcggcctcccaaagtgctgggattacaggcgtgagccactgggcccggctgagaaccattgttttaaagaactgacttgaggagaagcagcaggatgccaggagaaagtcatgagatacaagaaggaatgctaagcaaggaaaatggtaaatgactaaaactgaaacaaacatggactgtataaaatgataaaaatgattataaaatcgagagtagaggtaaaaatgatatctacttgttcttgtttattagaaaatgtgtaacaatgtttagctgaagtaacatgtaagccacaaagggattgatttgagctaaggaattcaaagatgtctgtgttatttgccagaaggacagaagattgattatcttttaagtatccttgttaacacttaaggacaactactgcaagaatagaaatagaggatttaacttcaaaaccggtaaagaggaaataaatgagctgggagaaatcaccagttcaaaagacagcaagaaaggagtaagagtaaaaaatgggttaaatagataattacagaaaaagattgtagaatgaaattcaactatattgataacctataattaattaaaaggactataacattcctagttaagacatagacagtcaaactagattgaaaactaaaatctgcttagtatagaatagtagacatgaaaggtatagtctattcacaacaggcattactaatacctagggatagtaggagtgaaaattaaagaatggaaaaacatataccaggtaaataaaaaaaaagaaatggctaaagctatattaatttcagacaaaactgactttatggcaaaaagcattaagataatatttgactcagataaggtgcatcacttgctggtgacagggtactaaagcctgaatgtatgtaaatataatctgcatttcttatcctaagacttcttcatagtcctagccctcaggtgtgaatttctggcatagtgcatagcacacacaactcaaggagataccaaatacaatccactgctaaaaaccagtaaaatacataagatagtagtagataataaaagagtgaataccttatttcttaaaactgaaaaaatgcctctttggtttttctcagccacataccctattacagagagtcaaagcctgcacaacagaagaggatcaggagaagctgatgcaaattaccagtctgcattcactgaatgccttcttgctgccaattaaaactgtaggtgtgcaggtgagttgtgtgaattcattcaccagtgatatacttttttttttttttcttttcttgagacggagtctcactgtgttgcccaggctggagcgcagtggcgtgatctcagctcattgcaacctccgcctcctgggttcaagcactctttctactaatcttgaaataaggattacttaggaaaatgaaatatgtgagtatatataccaagtttagagaaataaggtaattttttatagaatgtttagggagtgaatagttaatggaactaaatagtcaggctgagattttgtagttttggaacatctatgaactctaaccaaaggtaaatattgaaagcaaccctttcctatagggttggcatgcttacttctgccttgtagggcagggatcaagaggtcatttatcctaggtgtgaaatatactgtggttttagaagtctttaactgcaggaaggcctccagatgagtaagttacaccatttaggtgttgttctcagcttgtgtattccctgtactagggaacacattctactgatgttgtgtttattctgcctttctagttgtaaaagaacatgagaataatatagttctcaattataaaatgctacttgcaaaatgactaataactaataagatagtatgttaatttctcctcttttttctggagacagggtcttgccctgtcgcccaggctggagtgcagtgatgtgatcatggcgtactgtagccctgaattcctgggttcaagcgaccctactacctcagcttcccaagtagctgggactacaggcaagcaccatcatgcctggctaatttttaaatttttttgtagagatagggtctccctgtgttgctcaggctggctttgaaattctggcctcaagtgatccttcactttggcctcccaaagtgttaggattggaagcatgagacactgtacccggccttctttatatgatcctcaaatgagtttttggggagcaaaagtgtgggagggtagggaagagaaaggttattttcttctatttcaagtttgtggttaaatggagacactagaagaggtgtcagtagttcaactcgaaaatggtataagtgccagttattgggttaatgtgtttataatgtcctcttacagtaaaattatgtgtagaatatacatatgtaactaacctgcacattgtgcacatgtaccctaaaacttaaagtataataataaaaaaataaaaaataaaaaaaaagaaattctgtttcagagattattcttctaaaacctaataagcattttttctaaatatatgaatctgtcagtttaaattcccctttgctctgaattcatgcagttctgcctgtaaataaaacaagataaaaattgattgttgccaggcgcagtggttcatgcctgtaatcccagcactttgggaggctgaggcaggcggatcacctgaggttggaagttcaagagcagcctgaccaacatgcagaaaccccatctctactaattagccgggtgtggtggtgcatgcctgtaatcccagctagtcgggaggctgaggcaggagaattgcttgaacccaggaggcggaggttgcggtgggccaagattgcgccattgcactccagcctgggcaacaagagtgaaactccttcttaaaaaaaaaaaaaaattgattgttatttttaaagtaaactaaaatattttatttcatgaaagtgatagagccagctattgggttaaggaactcttgccttctttttaaaggccaaatgttgtaagctgcttatgttgttttaaaatcactattgaaataaccttacttggagttactgatactttctttcagtggtttatggattatctcattcatacataaatttattttgaaatttgcaatgatacttacaggcagtattaccctaatatataaaaatagaattgtggacaggttatttaattcttaaaagtttctgagttttaagtagatttgttatagagaacttcaacgtctgtgtgctaactgctgctacaaagggagttgaaatagttccccactcttctttttagagaagtgttacaaaaaatagatacttgagataatctgatgaatgagtgatttctttcttgaaaatgaaacttattcaggctctactacttttcccaccaaaataaaattttcttttgttattggataactgactttagtctcttaccctttgaagcttagacccttgcaagcccagataaaaatgatggttcatagttagtataacaatgcattttattttctaaataccaaagtaaaccgtatttgtaaaaaatgaaaatacattaaaaaaaattctttgtatttctactctccagttaagttttgtatgtgtctttccagaaacactttaaaaagaaaataaaagaggggatttggggtacacattgtcaacttttttcttttaactgaacatcatgaacttacctttctgtgcctttccaagtagatggttcttaactgttccctcatattctatgcataccatcatttatttaactattcttctaaccaatgggcatttaaactgttctgttttttattattataggcatcccatcagttatccatgtatttatgtaggatcattttatgtgcatgtgcattttacactttttttttttttttttttttttttgagatggagtcttgctctgtcacccaggctggagcacagtggcttgatctcagctcatggcaacctccacctaccaggttcaaacgattctcctgttgcagcctcctgtgtagctggggttacaggcacacgccgccatacctggataattttttgtattttagtagagacggggtttcaccacgttgcccaggttggtctcgaactcctgagctcaggcaatccacccgcctcggcctcccaaaagtgctaggattacaggcatgagctaccacgcccagcccattttacacttttatagatgctatcacattagcattcacatatgttgaattttcacattcttagtacctgccactttgggcattaggtacttctctaatctgttactttttaaaattgcttttcataaaaaaagcaaattacttggattaaacaggacacaggaaaagagaataaattatcagttttatttacttattttaaaattaactttttttttttattatactttaagttttagggtacatgtgcacattgtgcaggttagttacatatgtatacatgtgccatgctggtgcaccgcacccactaactcgtcatttagcattaggtatatctcccaatgctatccctcccccctccccccaccccaccacagtccccagagtgtgatattccccttcctgtgtccatgtgatctcattgttcaattcccacctatgagtgagaatatgcggtgtttggttttttgttcttgcgatagtttactgagaatgatgatttccaatttcatccatgtccttacaaaggacatgaactcatcattttttatggctgcatagtattccatggtgtatatgtgccacattttcttaatccagtctgtcattgttggacatttgggttggttccaagtctttgctattgtgaataatgccgcaataaacatatgtgtgcatgtgtctttatagcagcatgatttatagtcgtttgggtatatacccagtaatgggatggctgggtcaaatggtatttctagctctagatccctgaggaatcgccacactgatttccacaatggttgaactagtttacagtcccaccaacagtgtaaaagtgttcctatttctccacatcctctccagcacctgttgtttcctgactttttaatgattgccattctaactggtgtgagatggtatctcatagtggttttgatttgcatttctctgatggccagtgatgatgagcattttttcatgtgttttttggctgcataaatgtcttcttttgagaagtattgatgggacatatctcaaaataataagagctatctatgacaaacccacagccaatatcatattgaatgggcaaaaactggaagcattccctttgaaaactggcacaagacagggatgccctctctcaccactcctattcaacatagtgttggaagttctggccagggcaattaggcaggagaaggaaataaagggtattcaattaggaaaagaggaagtcaagttgtccctgtttgcagacaacatgattgtatatctagaaaaccccactgtctcagcccaaaatctccttaagctgataagcaacttcagcaaagtctcaggatacaaaatcaatgtacaaaaatcacaagcattcttatacaccaacaacagacaaacagagagccaaatcatgagtgaactcccattcacaattgcttcaaagagaataaaatacctaggaatccaacttacaagggatgtgaaggacctcttcaaggagaactacaaaccactgctcaaggaaataaaagaggatacaaacaaatggaagaacattccatgctcatgggtaggaagaatcaatatcgtgaaaatggccatactgcccaaggtaatttacagattcagtgccatccccatcaagctaccaatgactttcttcacagaattggaaaaaactaaagttcatatggaaccaaaaaagagcccgcatcgccaagtcaatcctaagccaaaagaacaaagctggaggcatcacactacctgacttcaaactatactacaacgctacagtaaccaaaacagcatggtactggtaccaaaacagagatatagatcaatggaacagaacagagccctcagaaataacgccacatacctacaactatctgatctttgacaaacctgagaaaaacaagcaatggggaaaggattccctatttaataaacggtgctgggaaaactggctagccatatgtagaaagctgaaactggatcccttccttacaccttatacaaaaatcaattcaagatggattaaagatttaaacgttagacctaaaaccataaaaaccctagaagaaaacctaggcattaccattcaggacataggcatgggcaaggacttcatgtccaaaacaccaaaagcaatggcaacaaaagccaaaattgacaaatgggatctaattaaactaaagagcttctgcacagcaaaagaaactaccatcagagtgaacaggcaacctacaaaatggaagaaaattttcgcaacctacttatctgacaaagggctaatatccagaatctacagtgaactcaaacaaatttacaagaacaaaacaaacaaccccataaaattaacttttaagctgatacttaaacacgttgataagtaaatgtttatgaattgtttttgtctcccacttaagaattctttatccactcaaaaaacacaggcagatcttgtctgccaaattataatttttcataattttattttatttttttgagatggggtcttactctgttgcccaggctagagtacagtggcatgatcactgctcacgatagcctcgacctcgttgggctcaggtgatcctcctatctcagcttcctgagtagcagggactacaggtgcatgccaccacactgcccagctaatttttgtatattttgtagagacagggtgttgccatgttgtccaggctggtctcaaacttctgggctcaactgacccacccacctcggcctcccaaagtgctaggattataggtgtgagcttctgtacctggccttgtctgccaaattaaatatgctggttatccacagttgtgcaggtaatgggaatgttgaatactttttttttttttgagacagagtttcactctgtcacccaggctggagtgcagtgatacgatctctcagctcactgcaacctccacctcccaggatcaagcagttctcctgcctcagcctcccgagtagctgggactacaggttcatgccaccatgcctggctaatttttgtatttttagtagagatggggtttcatcatgttggccaggctggtcttgaactcatgacctcaggtgatctgcccacctcggcctcccaaagtgctgggattacaggcatgaaccactgtgcccagcctaaattcttgatattaccatgatactttatgtatgtcttagctgattcaggtaggatttaatatgggattttaatttggaatttatttataccattaaagaagcatttagtatatactttcattcaaaatttccaaaaatccattaataatgtcaccatgcataagatatttttcaatgcatcttttcttttttttttaaaaaaaaaaaaaagctttgtcaagttaaactggtgtatcttttgactattaaaaattattgattatctttttattttcagggtgactgtcgttcctacagttacgtgtgtggaatctccagtaaagatgaacctgactgggaatcacttatttttctggctaggcttatacctcgcatgtgtcacaacgttaacaggtgtgtttcacaggagctagggtgggggcttgtgtaatggaaggatgtatttcttaagggctttcaattaatggaagaccaaaaggctcaaatgtagatataggtaggtcatgtagataagagtaaatggctgtgtttgtaattcaaatatgcacataggttgcagttaagtagatatttaatattatcttgtgttttgactcttcatcacaatgtagtcatctgttaaaaattatttaaaggagaaagatttttattttagtgtaaaatattaaaggcagctgtaaaacatcttctaaattagtggtttatattagctgttggttttatggtaacatgttttattccagaaggattgaaaaaagtatgtccaggttacagaattacaaataaagtaacacacacacacacacacacacacacacacacacacacccctacaaaacaaacaacccccactccaaacaaaaatggagacttggttaatttagtagtaaccccaaggaaaaaaatcaaagaaaagaaataggcatgtgccagatagcattaaaatagttacatttaaatatataatttacatttagattaatttccctttaatttgcatattaacttcttacattggatatatcaacagctgatttataatctatttctaatcctcaatttcatcgtaccatatatagttcaaaaattcacttattttgtactagattgctaattaaatgttcttacttcacagttattctctctgttcagttttcttcatgggtgttaggcagttaacaaccaaaaagaattacttgaggtttcattttactcaaattgctgcagaaatcagaagcttaccttttaatgttaagctagcagagtctgtatagttcgagataaggagttacagggaagccatgtttttaccgatgggaacatgctcattcaaaggcttactcaaatgaactgaaaaccgtcaaatacagaagcatctttaaaatgctgaaaaatagaattattactatcttcaataatgggggccaggcatggtggctcacgcctgtaatcccaccactttgagaggccaaggcgagtggatcacttgagcccaggagtttgagaccagcctgggcaacatggtgaaactccatctctacaaaaaatacaaaaattagccaggcatggtggtgtgtgcctgtagtcccagctactcgggaggctgaggcagaagaatggtgtgaacccgggagacagagcttgcagtgagctgagattgcaccactgcactcaagcctgggcgacagagcgagacttcatctcaaaaaaaggaaaaagaaaaaaaaaagaatcttggtgaactttagatttatttgctggggtctttttgatgaattggagtttaaaaatattcttccctgtttgccggtttttttcctaatttggataagggaatctttgtacagggagggactgttccagttttactgtcatatatagtgtgggtaaatacctgtcccagtacactctagtgattacttttgtttgtaagaaatgagaaggtgaaaagagcaggttcatggcattctcttaactttctcctgtaaagatcttttatgaactttaggaactgtattgtttagaaaaattataacttactgtctatacgtgggacaaaaaccttgcttctgttgtagtatgtttggaaggagacaataaagtagttggtgataccggtggttttgtgctaatatggttttctattgtgtattttacctaagtacatattaagaaaagaagaaaaaacattaaactttaccatgatagtttccggggagatttattttctttgctaaaaacaggactaacgaaaagtgaatagggagaactagctaagtggcactcatatctctaccctgattccctcttgttttctgggtgaagattgtgaaacagatcccttcaaaccagtggccagctacacttcaagttgttaaaattgtctgattctaatttcctcaagttatgttccaaactgtactctcattaactcaagatatttgtggtgtatcttcatgtctttcaaacagacatttatataaaacactaatgaatcatttttttggaaggaagtagtaggatactaagtcctatttcaatggcctttttaatgaagtgcaaatgagaatctctctagaatgattgtttctcaagtagagaaaaaaagatgagtttactaattttgggaaattcactgtagtttcctcttggagatttccagtgcatgttagcatagtatattaatagtttctgaagtcttgtcataaagacacctatttttaaattttaattgtgtatttgtttcctaggactacccataacaaatcaccacaaactaggaggcttaaaacaacagacatttattctctcacagtttaggaggccagaagtctgaattcagtgttggcagggtcatattctctctgaagactctaggggaggatccttccttgcttcttcctactatcgagtggtggcagcatagtccagtctgtctttgtgttcacatgaccctcattcccctgtgggtatctgtgtatccaaatttcccagcacttggaaggataccagtcattggattagagcccatcctaatccagtattacttcattttaacttgattacatttgcaaagaccctttttctccccagagattctgggtgggcatgaagttttggggatactcttcgacccagtacattaagtgaaaagagagttgtaataatcacttatatctttgcacattctgagatacagaatattccagagaacacactttgaggaaaacacgtagaacacagaccttcctataatcttatatcttgtcctatccatctggggagttgaagtgggcaggagaagggcctgaaactaagtgggaaatggaaagaatgttactgttctaagtgccttgcttctcatactaaaatgtaatttctacatttgaaatgtaattgctgtattatttttacctctttgtttccagagttgtttatatatttggcccaccagttaaagaacctcctacagatgttactcccactttcttgacaacaggggtgctcagtactttacgccaagctgattttgaggcccataacattctcagggagtctggtaagttgctcatgtttttgattactaccctctgaaactagtttttggaagcttgatattaagagtaggaggatgtggcttttgctttttagatgcttttgtttttattatttaaatctttgaatgttctatgatgttgcttttttttcttgagacggagtctgtcttgctctgtcgcccaggctggagtgcagtggcacaatcttggttcacttcagcctctgcctcctgttacaagtgattctcctgcctcagcctcccgagtagctgggattacaggcacccgccaccacacctggctagttttttgtatttttagtagagatagggttttgccattttggccagctgatctcgaactcctaacctcatgtgatcaggtcaggagtcaggtcaggccagccttggcctcccagagtgttgggattacaggcgtgagccactgcacctggcctataatgttgcttctaaataaaatatattcccctttgtggccatttgtggacatgcacagagcagcagaaaatttgaatttccagacttagatgttcccagctgagtttgaacaaggtgacgctatgccttcttgttttcagctctcataatgtgaacaagtctttttcatggtctccttgatgctatatcttttgcatttttatgtcttttgttggtgattttgctgtttaaaatggatctcaactatagtgttgaagtgctgtctgggttcctaagcacaggaatgctgtgatgtaccttacagagaaaatctgtgtgttagataaacttcattcaggcatgagttaaagagctactgattgtcagttcagtgttaatgaatctataataaatattaaatacagtgtcttaaatcagaaacacataaaacaaggttatatattaatcagttgatgaaaatattacaaccagaggcttgcaggaacctaaccctccatttcccctgggagaaatggttcagtattcgctaattcgttgtttgcagtgtagaatataactatactgcaaattataatagagaataatgagactggactgtttctgtttacagcacatcagtgaaacatcactgatgtgatcaaattctgttttttgcatttgtgattaagtccatttaaaatgctctgcgagtagctgaaatcaatgcatgatgacagtcttaactacgctgtgtgtgtatatgtgatttcagatattgcctgggaggatttaattgaactgagttagagtgcttttctatggtgtggtgattggttctacatatacctttctctagggtatgctgggaaaatcagccagatgccggtgattttgacaccattacattttgatcgggacccacttcaaaagcagccttcatgccagagatctgtggttattcgaacctttattactagtgacttcatgactggtatacctgcaacacctggcaatgagatccctgtagaggtaatttatatatttttttctaatgcacgttctcagtacctcttacattttataatatggtgaatggaataggctatgccagtgctgtatttcttatgttggttttctgttcataagataggcttttttttttaaattttcacatatttgattaatacactggggcagaaaaaggctttaaattatatatgctctccaattgtgtgattactgtataaaaagtaattgattataattttatctaatcctgccaatttctactttacaaacagtattccccaaataaaaaagaaaactctaggagtgaagaagagtttgttagaaataaggatgcaatttgatagctttatggccagacacccaagaattgcctaaatgttctccctactaccaattccaactcaatttttgatccttctttgtcttcaccagaacattccaaaggagctacttgcataatagcaagtgaaaatcaaagtgagactctattagttgggccactgcctccagtgcactaacacgaagctgtcgtgatagctaggtagatgggttggttcggaacgtggcttagagtcatttttctcttaggctcaaatttgtcaaatttgacaagtgagcaggctatcctctcctgtatacttgataatgattattacagtgtactctgtgccaccttggtggcccttagagttgtatgtttaagctgcagataatttgcagagcattggctgaaacacataaaatcacatgcttggatgccacagcaatgtcaaatcgctatatgttcttaagcacttgtggctcagtcacaaacacaacttgcagactagcacctgtttgtggatcacactttgagtagtactgagtttaagtcactaatattttaaaccttaataacttacacctatccctgcagaggggaataggttcattatataagattaatcagaaaccatcccatatacatggaggacaatagtgaaaatagtcaaattttaatttcaaattacaaatgtaagccctctaggactgctggacatgcagtggatgctgacttttctctataattttttttaataggtggtattaaagatggtcactgagattaagaagattcctggtatttctcgaattatgtatgacttaacatcaaagcccccaggaactactgagtgggagtaataaacttcttgttctattaaagtaccgtgtgcagtttaaattgattagaaatcattcccattattgacatgcagtactgtgaaaagagttactggagcagctacatcacatctgagttctccacagcaaaaatctacggcttaagagctgagttggggataaccaaaagggactgaagagtttgtacgggtaaataatcaaagcaccattgcctacactcagacagattgatactgcttttgcctttgcctcacttattctttatgtataaattcactgttgatgtctctgtgaatatgtatgaaggtgggtgaatttgggggtaattatgggcttctgtctccttaccagtttctaagaactgttagaaacgcccattatttaccagtgttacttactacttatatatactttttggagaatcagttttcttataagtaatcttatttctccaaagggtgaaaaagagatgaacatatcagataaaggtataggtgggctattccagccacttttgaaggacactgagggtataaaatgtaagctaccataagatggaaaagcaccaggaatttgttgatgtacttttgaattttatttcacagccttgggaggaagaaactatgagtggtctcagatgtctatgaatggtcttaaaatgttttcaaaccatgttccacatagatgccatgttattcaatgtcagcttgtttattagtaaggagctttatcctcctagccttatattaccagccccccacagccttgcatagaaagtttctgttatctccttagaagaaactggctcaatgacgtttctggtgtagtgcagatagaggtcaagaggagcattttcatctcttcttttgctggtcagagaaacggttctgagatcacagaagctatgatgcatacctacagcttacaaaagggagaggaaaaatgtttatatcatctagtaaacagttgtaaaatgagcttttaaatctaatctatagacatattattcattgctcag

SEQ ID NO: 2 - DNA, 150 bp, Homo sapiens
  mutation (92)..(92): T>C
cttcggtgccgccagcctcccgaagtcatccaaggtagcaggcaaatccggcagcagcaaactagctcggattcggcttcggtttttctcccggccggcgccacaggcccctcctccggggcgggctcctcggtgcgagggcaggagggg

SEQ ID NO: 3 - DNA, 151 bp, Homo sapiens
  mutation (118)..(118): InsC
cttcggtgccgccagcctcccgaagtcatccaaggtagcaggcaaatccggcagcagcaaactagctcggattcggcttcggtttttctcctggccggcgccacaggcccctcctcccggggcgggctcctcggtgcgagggcaggagggg

SEQ ID NO: 4 - DNA, 116 bp, Homo sapiens
  exon (1)..(116): EXON 13
ggtgactgtcgttcctacagttccgtgtgtggaatctccagtaaagatgaacctgactgggaatcacttatttttctggctaggcttatacctcgcatgtgtcacaacgttaacag

SEQ ID NO: 5 - DNA, 116 bp, Homo sapiens
  exon (1)..(116): EXON 13
ggtgactgtcgttcctacagttacgtgtgtggaatctccagtaaagatgaacctgactgggaatcacttatttttctggctaggcttatacctcgcatgggtcacaacgttaacag

SEQ ID NO: 6 - DNA, 27 bp, Artificial Sequence
  Description of Artificial Sequence Synthetic primer
cgccagcctcccgaagtcatccaaggt

SEQ ID NO: 7 - DNA, 20 bp, Artificial Sequence
  Description of Artificial Sequence Synthetic primer
gcccttgggaggaggagccc

SEQ ID NO: 8 - DNA, 24 bp, Artificial Sequence
  Description of Artificial Sequence Synthetic primer
aactggtgtatcttttgactatta

SEQ ID NO: 9 - DNA, 25 bp, Artificial Sequence
  Description of Artificial Sequence Synthetic primer
cattaattgaaagcccttaagaaat

SEQ ID NO: 10 - protein, 693 aa, Homo sapiens
MALCNGDSKLENAGGDLKDGHHHYEGAVVILDAGAQYGKVIDRRVRELFVQSEIFPLETPAFAIKEQGFRAIIISGGPNSVYAEDAPWFDPAIFTIGKPVLGICYGMQMMNKVFGGTVHKKSVREDGVFNISVDNTCSLFRGLQKEEVVLLTHGDSVDKVADGFKVVARSGNIVAGIANESKKLYGAQFHPEVGLTENGKVILKNFLYDIAGCSGTFTVQNRELECIREIKERVGTSKVLVLLSGGVDSTVCTALLNRALNQEQVIAVHIDNGFMRKRESQSVEEALKKLGIQVKVINAAHSFYNGTTTLPISDEDRTPRKRISKTLNMTTSPEEKRKIIGDTFVKIANEVIGEMNLKPEEVFLAQGTLRPDLIESASLVASGKAELIKTHHNDTELIRKLREEGKVIEPLKDFHKDEVRILGRELGLPEELVSRHPFPGPGLAIRVICAEEPYICKDFPETNNILKIVADFSASVKKPHTLLQRVKACTTEEDQEKLMQITSLHSLNAFLLPIKTVGVQGDCRSYSYVCGISSKDEPDWESLIFLARLIPRMCHNVNRVVYIFGPPVKEPPTDVTPTFLTTGVLSTLRQADFEAHNILRESGYAGKISQMPVILTPLHFDRDPLQKQPSCQRSVVIRTFITSDFMTGIPATPGNEIPVEVVLKMVTEIKKIPGISRIMYDLTSKPPGTTEWE

SEQ ID NO: 11 - protein, 38 aa, Homo sapiens
GDCRSYSSVCGISSKDEPDWESLIFLARLIPRMCHNVN

SEQ ID NO: 12 - protein, 38 aa, Homo sapiens
GDCRSYSYVCGISSKDEPDWESLIFLARLIPRMGHNVN

(19) United States
(12) Patent Application Publication
(10) Pub. No.: US 2010/0273152 A1
(43) Pub. Date: Oct. 28, 2010

(54) METHOD OF IDENTIFYING INDIVIDUALS AT RISK OF THIOPURINE DRUG RESISTANCE AND INTOLERANCE

(75) Inventors: Rebecca Lee Roberts, Christchurch (NZ); Richard Blair Gearry, Christchurch (NZ); Murray Lindsay Barclay, Christchurch (NZ); Martin Alexander Kennedy, Christchurch (NZ)

Correspondence Address: FOLEY & LARDNER LLP, P.O. BOX 80278, SAN DIEGO, CA, 92138-0278, US

(73) Assignee: University of Otago

(21) Appl. No.: 12/514,075

(22) PCT Filed: Nov. 8, 2007
(86) PCT No.: PCT/NZ07/00329

§ 371 Date: Jun. 15, 2010

(30) Foreign Application Priority Data
Nov. 8, 2006 (NZ) 551157

(51) Int. Cl. C12Q 1/68; C07H 21/04

(52) U.S. Cl. 435/6; 536/23.1; 536/24.3; 536/24.33

(57) ABSTRACT

The present invention is directed to a method of screening individuals for the presence or absence of one or more polymorphisms associated with the risk of thiopurine resistance or intolerance.

### FIELD OF THE INVENTION

The present invention relates to methods and kits for identifying individuals at risk of thiopurine drug intolerance. These methods and kits are based on detecting the presence of polymorphisms in the GMPS gene associated with thiopurine drug resistance or intolerance.

### BACKGROUND TO THE INVENTION

Thiopurine drugs have been used to treat a number of diseases. Amongst these are acute lymphoblastic leukemia, Inflammatory Bowel Disease (IBD), complications associated with solid organ transplantation, rheumatoid arthritis, dermatological conditions and autoimmune conditions. A number of these conditions are also on the increase in the population.

Thiopurine drugs are metabolised in the body and the active metabolites 6-thioguanine nucleotides (6-TGN) are produced. Unfortunately, up to 40% of individuals demonstrate drug resistance or intolerance to treatment using thiopurines. A proportion of individuals that are resistant to thiopurine treatment are unable to achieve therapeutic levels of 6-TGN, and instead accumulate 6-methylmercaptopurine ribonucleotides (6-MMPR) to hepatotoxic levels (>5700 pmol/8×10⁸ RBC).

Guanosine 5′ monophosphate synthetase (GMPS) is an enzyme naturally involved in de novo synthesis of purine nucleotides, and it is one of several enzymes involved in the metabolism of thiopurine drugs. Prior art relating to the GMPS gene include its cloning and expression for use in screening of inhibitors of the GMPS enzyme (U.S. Pat. No. 5,789,216), and its involvement in a chromosome translocation associated with treatment related acute lymphocytic leukaemia (Pegram et al, 2000).

Research in the field of thiopurine resistance or intolerance has focussed on thiopurine S-methyltransferase (TPMT). A link is known to exist between levels of 6-TGN and polymorphisms in TPMT. U.S. Pat. No. 5,856,095, for example shows genetic polymorphisms and the relationship with thiopurine intolerance (myelotoxicity) or resistance. However, this link does not account for thiopurine resistance or intolerance in a significant proportion of individuals.

An assay or method that provides an improved or alternative means of identifying individuals at risk of thiopurine resistance or intolerance would be useful to practitioners attempting to establish such a risk in individuals in need of thiopurine therapy.

It is therefore an object of the present invention to provide methods for kits for identifying individuals at risk of thiopurine resistance or intolerance or to at least provide the public with a useful choice.

### SUMMARY OF THE INVENTION

The present inventors have surprisingly discovered that there are a number of polymorphisms present in the guanosine 5′ monophosphate synthetase (GMPS) gene that are associated with individuals response to thiopurine therapy. More particularly, polymorphisms in the promoter region and the coding region of GMPS have been identified as being associated with a risk of drug resistance or intolerance to thiopurine therapy in individuals undergoing such therapy.

In this specification, positions are indicated with reference to SEQ ID NO: 1 unless the context indicates otherwise. Four specific polymorphisms are identified herein. Two polymorphisms are located in the promoter region of GMPS at position 692 where T becomes C and at positions 717 to 718, where a C is inserted. Two further polymorphisms are located in exon 13, where at position 62120, A becomes C, and at position 62197, T becomes G.

In a first aspect, the present invention provides a method for screening individuals for the presence or absence of one or more polymorphisms associated with the risk of thiopurine resistance or intolerance, which method includes the step of determining the genotypic state of the individual with respect to the GMPS gene.

The genotypic state may be determined with respect to DNA or mRNA (for polymorphisms in the coding region) obtained from said individual, by direct or indirect methods. By direct methods is meant that the polymorphism per se is detected, and by indirect methods is meant that the presence of the polymorphism is determined by detecting the presence of a linked polymorphism. Preferably, the linked polymorphism is in linkage disequilibrium with the polymorphism of the invention.

Preferably a biological sample containing DNA is obtained from an individual and the genotypic state of the GMPS gene assessed for the presence of at least one nucleotide difference from the nucleotide sequence encoding GMPS (SEQ ID NO: 1), either by direct or indirect methods.

More preferably the genotypic state is determined by the presence of one or more polymorphisms selected from SEQ ID NOs 2 and 5, either by direct or indirect methods.

In another embodiment the invention provides a method of identifying an individual at risk of thiopurine resistance or intolerance, said method comprising:
obtaining a biological sample containing nucleic acids from said individual and identifying a polymorphism selected from the group consisting of SEQ ID NOs 2 to 5 of the GMPS gene, wherein the presence of said polymorphism is associated with a risk of thiopurine resistance or intolerance.

In still a further aspect, the present invention provides an isolated nucleic acid molecule suitable for use in detecting a polymorphism selected from the group consisting SEQ ID NOs 2 to 5 of the GMPS gene, said nucleic acid molecule consisting of a nucleotide sequence having about at least 15 contiguous bases of SEQ ID NO 1 or a complementary sequence thereof.

In one embodiment, the nucleic acid molecule consists of a probe having a sequence which binds to the nucleotide sequence which contains at least one polymorphism.

In another embodiment, the nucleic acid molecule consists of a primer having a sequence which binds to the GMPS gene either upstream or downstream of a polymorphism. The primer in a preferred embodiment binds to the GMPS gene sequence upstream or downstream of a polymorphism and up to one base from said polymorphism.

Preferably, the polymorphism is located in the promoter region or in a coding region of the GMPS gene sequence. More preferably, the polymorphism is located in the promoter region at position 692 where a T is replaced by C and/or at position 717 to 718, where a C is inserted between the nucleotides at these positions.

Where the polymorphism is located in a coding region of the GMPS gene sequence, it is preferably in exon 13 at position 62120 where A is replaced by C, and/or at position 62197 where T is replaced by G.

In a still further aspect, the present invention provides an isolated nucleic acid molecule having the sequence of SEQ ID NO: 1 and comprising one or more polymorphisms selected from the group comprising SEQ ID NOs 2 to 5, or a fragment, variant or antisense molecule thereof.

The nucleic acid molecule may alternatively comprise peptide nucleic acid (PNA). The nucleic acid may further comprise a detectable label, preferably a fluorescent label. Alternatively, detection may be accomplished by use of radioisotopic labels, or by methods that differentiate mass of reaction products.

In a further preferred embodiment, the nucleic acid molecule contains two polymorphisms comprising SEQ ID NOs: 2 and 3 or SEQ ID NOs: 4 and 5.

In another aspect, the present invention provides a diagnostic kit for identifying individuals at risk of thiopurine resistance or intolerance based on assessment of the genotypic state of the GMPS gene.

In a preferred embodiment, the kit comprises a probe of the invention.

Alternatively, the kit comprises a primer that binds to the GMPS gene or the antisense strand thereof up to a nucleotide positioned one base from a polymorphism. The primer may be upstream or downstream of said polymorphism.

In a further aspect, the present invention provides a diagnostic kit for identifying individuals at risk of thiopurine resistance or intolerance comprising first and second primers which are complementary to nucleotide sequences of the GMPS gene or the antisense strand thereof upstream and downstream, respectively, of at least one polymorphism.

Preferably the at least one polymorphism is selected from the group comprising at least one SEQ ID NOs 2 to 5.

The invention will now be described with reference to the sequences and figures of the accompanying drawings in which:

### Sequences

SEQ ID NO: 1 is the genomic sequence for the GMPS gene as generated by UCSC Genome Browser (http://www.genome.ucsc.edu) (July 2003 Assembly);

SEQ ID NO: 2 is a partial genomic sequence from the GMPS promoter showing a polymorphism at position 692 in SEQ ID NO: 1. SEQ ID NO:2 corresponds to positions 601-750 in SEQ ID NO: 1. The polymorphism is at position −512 from the first coding region;

SEQ ID NO:3 is a partial genomic sequence from the GMPS promoter showing a polymorphism at positions 717-718 in SEQ ID NO: 1. SEQ ID NO:3 corresponds to positions 601-750 in SEQ ID NO: 1. The polymorphism is at positions −486 to −487 from the first coding region;

SEQ ID NO: 4 is a genomic sequence of GMPS exon 13 showing a polymorphism at position 62120 in SEQ ID NO: 1. SEQ ID NO: 4 corresponds to positions 62098-62213 in SEQ ID NO: 1. The polymorphism is at coding position 1583 in the processed DNA molecule after introns are removed; and

SEQ ID NO: 5 is a genomic sequence of GMPS exon 13 showing a polymorphism at position 62197 in SEQ ID NO: 1. SEQ ID NO: 5 corresponds to positions 62098-62213 in SEQ ID NO: 1. The polymorphism is at coding position 1660 in the processed DNA molecule after introns are removed.

### BRIEF DESCRIPTION OF THE FIGURES

FIG. 1A is a schematic of the 181 by fragment generated by PCR which encompasses both GMPS promoter SNPs, indicating the restriction enzyme sites for BstNI (specific for 692T) and SmaI (specific for 717 to 718 insC);

FIG. 1B is a resolution of the above PCR products on a 3% agarose-TBE gel;

FIG. 2A is a schematic representation of the 243 bp fragment generated by PCR which encompasses both GMPS exon 13 SNPs, indicating the restriction enzyme site for BsaAI (specific for 62120A);

FIG. 2B is a resolution on a 3% agarose-TBE gel of the above PCR products, digested with BsaAI;

FIG. 2C is a schematic representation of the 243 bp fragment generated by PCR which encompasses both GMPS exon 13 SNPs, indicating the restriction enzyme site for BslI (specific for 62197G); and

FIG. 2D is a resolution on a 3% agarose-TBE gel of the above PCR products, digested with BslI.

### DEFINITIONS

The term “drug” as used herein refers to a chemical entity administered to a person in a medical context to treat or prevent or control a disease or condition.

The term “therapy” refers to a process which is intended to produce a beneficial change in the condition of an individual. A beneficial change can, for example, include one or more of: restoration of function, reduction of symptoms, limitation or retardation of progression of a disease, disorder, or condition or prevention, limitation or retardation of deterioration of an individual's condition, disease or disorder.

In the context of the present invention, “thiopurine therapy” involves the administration to an individual of a thiopurine drug. Non-limiting examples of thiopurine drugs are azathioprine (imuran, azamun, thiopurine) and 6-mercaptopurine (puri-nethol).

In this specification “thiopurine intolerance” means an adverse reaction, such as liver toxicity, in individuals undergoing thiopurine therapy.

“Thiopurine resistance” means a lack of a desired therapeutic outcome in individuals undergoing thiopurine therapy.

“Individual” means a human being.

“Biological sample” as used herein means any sample derived from an individual to be screened. The sample may be any sample known in the art in which the GMPS gene can be detected. Included are any body fluids such as plasma, blood, saliva, interstitial fluid, serum, urine, synovial, cerebrospinal, lymph, seminal, amniotic as well as tissues such as liver and kidney.

“Polymorphism” in the present invention means a variant form of a gene with reference to one or more positions in the gene sequence, whether or not the polymorphism is in the coding or non-coding portion of the gene. It also includes synonymous and non-synonymous polymorphisms in the coding region of a gene. One common class of polymorphisms, relevant to this application, is referred to as “single nucleotide polymorphism” (SNP), which refers to the occurrence of a different nucleotide at equivalent positions in different individuals.

“Nucleic acid molecule” as used herein means a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified nucleic acids. Reference to a polynucleotide is to be similarly understood.

The term “isolated” as applied to the nucleic acid sequences disclosed herein is used to refer to sequences that are removed from their natural cellular environment. An isolated molecule may be obtained by any method or combination of methods including biochemical, recombinant, and synthetic techniques. The nucleic acid sequences may be prepared by at least one purification step.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it, is operably linked to promoter and terminator sequences and/or other regulatory elements.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors.

“Primer” refers to a single-stranded nucleic acid molecule, also referred to as an oligonucleotide, which specifically hybridizes (binds) to a predetermined region of DNA of complementary sequence. Primers are key reagents in polymerase chain reactions (PCR), and in a variety of polymorphism detection methods. They provide specific initiation sites for the enzymes used in PCR and in many polymorphism detection methods.

“Probe” refers to a nucleic acid molecule which detectably distinguishes between nucleic acid target molecules differing in sequence. Detection can be accomplished in a variety of different ways depending on the type of probe used and the type of target molecule. Thus, for example, detection may be based on discrimination of binding affinity of the target molecule, but preferably is based on detection of specific binding. One example of specific binding is nucleic acid probe hybridization. Thus, probes can include nucleic acid hybridization probes.

“Specifically hybridizes” indicates that a probe hybridizes to a sufficiently greater degree to the target sequence than to a sequence having a mismatched base at least one polymorphism to allow distinguishing such hybridization. The term “specifically hybridizes” thus means that the probe hybridizes to the target sequence, and not to non-target sequences, at a level which allows ready identification of probe/target sequence hybridization under selective hybridization conditions.

Thus, “selective hybridization conditions” refer to conditions which allow such differential binding. Similarly, the terms “specifically binds” and “selective binding conditions” refer to such differential binding of any type of probe, and to the conditions which allow such differential binding. Typically hybridization reactions to determine the status of polymorphisms in individual samples are carried out with two different probes, one specific for each of the (usually two) possible polymorphic nucleotides. The complementary information derived from the two separate hybridisation reactions is useful in corroborating the results. Such hybridisation generally occurs under stringent hybridisation conditions.

“Stringent hybridisation conditions” takes on its common meaning to a person skilled in the art. Appropriate stringency conditions which promote nucleic acid hybridisation, for example, 6× sodium citrate (SSC) at about 45° C. are known to those skilled in the art, including in Current Protocols in Molecular Biology, John Wiley & Sons, NY (1989). Appropriate wash stringency depends on degree of homology and length of probe. If homology is 100%, a high temperature (65° C. to 75° C.) may be used. If homology is low, lower wash temperatures must be used. However, if the probe is very short (<100 bp), lower temperatures must be used even with 100% homology. In general, one starts washing at low temperatures (37° C. to 40° C.), and raises the temperature by 3-5° C. intervals until background is low enough not to be a major factor in autoradiography. The diagnostic kit can also contain an instruction manual for use of the kit.

“Genotyping” or “Genotypic state” refers to a range of methods, including those reviewed by Kwok (2001), which determine the nature of the alleles at a polymorphism in the DNA of an individual.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

### DETAILED DESCRIPTION OF THE INVENTION

Effective treatment of inflammatory bowel diseases such as Crohn's disease and ulcerative colitis with the thiopurine drugs 6-mercaptopurine and azathioprine (AZA), is complicated by the toxic side effects in patients who are resistant or intolerant to this therapy. Such toxic side effects including allergic reactions, neoplasia, opportunistic infections, vomiting and nausea, hepatitis, bone marrow suppression and pancreatitis. Therefore, it is critical that thiopurine drug intolerance or resistance be measured in candidates for 6-mercaptopurine treatment in order to administer the appropriate dose or to avoid therapy in such patients.

The present inventors have found for the first time that polymorphisms in the GMPS gene are associated with thiopurine resistance or intolerance.

In the first aspect, the present invention provides a method for screening an individual for the presence or absence of one or more polymorphisms associated with the risk of thiopurine resistance or intolerance. The method includes at least the step of determining the genotypic state of the individual with respect to the GMPS gene.

The genotypic state may be determined with respect to DNA or mRNA (when the polymorphism is located in the coding region) obtained from said individual, by direct to indirect methods. As will be appreciated by art-skilled workers, where an RNA molecule is used, T in any DNA sequences should be replaced by U. By direct methods is meant that the polymorphism per se is detected, and by indirect methods is meant that the presence of the polymorphism is determined by detecting the presence of a linked polymorphism. Preferably the linked polymorphism is in linkage disequilibrium with the polymorphism of the invention. Linkage disequilibrium (LD) is a phenomenon in genetics whereby two or more mutations or polymorphisms are in such close genetic proximity that they are co-inherited. This means that in genotyping, detection of one polymorphism as present infers the presence of the other, (Reich DE et al; 2001). One or more polymorphisms in linkage disequilibrium with the polymorphisms specified herein can be identified, for example, using public data bases.

The presence of one or more polymorphisms in the GMPS gene has now been shown to be associated with a risk of thiopurine resistance or intolerance in individuals undergoing such therapy.

In some individuals, the presence of one or more polymorphisms has been shown to result in drug resistance, whilst other individuals may exhibit drug intolerance. There are some individuals who exhibit both thiopurine intolerance and resistance. By linking specific polymorphisms with therapeutic outcomes, a risk profile can be established to identify individuals at risk of thiopurine intolerance and/or resistance who have the same polymorphism.

Therefore, in another embodiment the invention provides a method of identifying an individual at risk of thiopurine resistance or intolerance, said method comprising:
obtaining a nucleic acid sample from said individual and identifying a polymorphism selected from the group consisting of SEQ ID NOs 2 to 5 of the GMPS gene, wherein the presence of said polymorphism is associated with a risk of thiopurine resistance or intolerance.

Determining polymorphisms enables records to be kept on the progress of individuals in thiopurine therapy. A polymorphism profile can therefore be established linking a probability with thiopurine intolerance or resistance with a particular polymorphism based on results from groups of individuals with identical or similar GMPS polymorphisms.

Using polymorphism profiles, individuals can then be more accurately assessed as to whether thiopurine therapy is likely to be effective. Where a low probability of successful therapy is found, alternative treatments can be used including methotrexate, infliximab and other biological agents. Alternatively, individuals may proceed directly to surgery if indicated.

Profiles can also be used to determine appropriate therapeutic dosage and frequency ranges for thiopurine therapy by comparing successful therapies of various dosage and frequency ranges in individuals with similar or identical polymorphisms.

Other risk factors can be combined into the analysis by a medical practitioner to support a prognosis of successful thiopurine treatment. These risk factors could be clinical or genetic and may include thiopurine methyltransferase (TPMT) enzyme activity or genotype and inosine triphosphatase (ITPA) enzyme activity or genotype.

An individual's genotypic state is determined by the presence of at least one nucleotide difference from the nucleotide sequence encoding GMPS (SEQ ID NO: 1) (determined by direct or indirect methods), and a grouping into the majority and a polymorphic minority is enabled permitting different probabilities for therapeutic success to be determined.

Polymorphisms in the promoter or coding regions of GMPS are more likely to affect an individual's probability of thiopurine therapy intolerance or resistance. However, intron polymorphisms, while less likely to affect the probability of thiopurine therapy intolerance or resistance, may nevertheless influence the probabilities of thiopurine therapy intolerance or resistance. Numerous examples of intronic polymorphisms affecting gene expression have been reported. The mechanisms underlying such effects are not fully understood, but amongst other possibilities intron polymorphisms can impact on normal splicing of mRNA leading to production of aberrant, possibly non-functional or reduced function transcripts.

Due to the redundancy of genetic code, synonymous polymorphisms are much less likely to affect expression levels. Nevertheless, such polymorphisms are considered to occasionally have an effect on expression of a protein due to the variable binding affinities of tRNA to different 3 base codons coding for the same amino acid. Synonymous polymorphisms therefore may still affect the probabilities of an individual demonstrating thiopurine intolerance or resistance.

The inventors have specifically identified polymorphisms in the promoter region and in the coding region. These are shown in SEQ ID NOs: 2 to 5. Where the polymorphism is selected from a change in the promoter region of GMPS, it may be selected from a change at position 692 where a T is replaced by C and/or from a change at position 717 to 718, where a C is inserted between the nucleotides at these positions.

Where the polymorphism is selected from a change in a coding region of GMPS, it is preferably from a change in exon 13, most preferably selected from position 62120 where A is replaced by C, and/or from position 62197 where T is replaced by G.

The specific polymorphisms identified herein indicate a higher incidence of thiopurine drug intolerance or resistance in individuals versus the genetic polymorphism of SEQ ID NO:1. In some cases, the polymorphisms specifically identified herein indicate up to a six fold increase in the probability of demonstrating thiopurine drug resistance or intolerance.

One method for identifying an individual at risk of thiopurine resistance or intolerance may comprise obtaining a biological sample containing nucleic acid from said individual. The sample is then analysed to identify a polymorphism selected from the group consisting of SEQ ID NOs 2 to 5 of the GMPS gene. If the sample indicates the presence of said polymorphism, the individual is associated with a risk of thiopurine resistance or intolerance. Preferably, the sample is whole blood and the sample is prepared for DNA analysis by known methods.

The methods of the invention are primarily directed to the detection and identification of the above polymorphisms associated with thiopurine intolerance or resistance, which are all single nucleotide polymorphisms. In general terms, a single nucleotide polymorphism (SNP) is a single base change or point mutation resulting in genetic variation between individuals. SNPs occur in the human genome approximately once every 100 to 300 bases, and can occur in coding or non-coding regions. Due to the redundancy of the genetic code, a SNP in the coding region may or may not change the amino acid sequence of a protein product. A SNP in a non-coding region can, for example, alter gene expression by, for example, modifying control regions such as promoters, transcription factor binding sites, processing sites, ribosomal binding sites, and affect gene transcription, processing, and translation.

SNPs can facilitate large-scale association genetics studies, and there has recently been great interest in SNP discovery and detection. SNPs show great promise as markers for a number of phenotypic traits (including latent traits), such as for example, disease propensity and severity, wellness propensity, and drug responsiveness including, for example, susceptibility to adverse drug reactions. Knowledge of the association of a particular SNP with a phenotypic trait, coupled with the knowledge of whether an individual has said particular SNP, can enable the targeting of diagnostic, preventative and therapeutic applications to allow better disease management, to enhance understanding of disease states and to ultimately facilitate the discovery of more effective treatments, such as personalised treatment regimens.

Indeed, a number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 1.5 million SNPs mapped onto the human genome sequence. Each dbSNP entry includes the sequence context of the polymorphism (i.e., the surrounding sequence), the occurrence frequency of the polymorphism (by population or individual), and the experimental method(s), protocols, and conditions used to assay the variation, and can include information associating a SNP with a particular phenotypic trait.

At least in part because of the potential impact on health and wellness, there has been and continues to be a great deal of effort to develop methods that reliably and rapidly identify SNPs. Initially, this was no trivial task, at least in part because of the complexity of human genomic DNA, with a haploid genome of 3×109 base pairs, and the associated sensitivity and discriminatory requirements.

There are many experimental methods well known and available to art-skilled workers for determining the presence of additional SNPs or other polymorphisms in the GMPS gene. These include, for example, methods based on denaturing high pressure liquid chromatography, DNA sequencing, chemical or enzymatic analysis of mismatched DNA or cDNA, and electrophoretic detection of mismatched DNA or cDNA.

The application of these methods to GMPS may provide identification of additional polymorphisms that can affect inter-individual probabilities of thiopurine drug intolerance or resistance. One skilled in the art will recognize that many such general methods have been described and can be utilized, as for example, reviewed by Syvanen and Taylor (2004).

To assist with detecting the presence or absence of polymorphisms/SNPs, nucleic acid probes and/or primers can be provided. Such probes have nucleic acid sequences specific for chromosomal changes evidencing the presence or absence of the polymorphism and are preferably labeled with a substance that emits a detectable signal when combined with the target polymorphism.

The nucleic acid probes can be genomic DNA or cDNA or mRNA, or any RNA-like or DNA-like material, such as peptide nucleic acids, branched DNAs, and the like. The probes can be sense or antisense polynucleotide probes. Where target polynucleotides are double-stranded, the probes may be either sense or antisense strands. Where the target polynucleotides are single-stranded, the probes are complementary single strands.

The probes can be prepared by a variety of synthetic or enzymatic schemes, which are well known in the art. The probes can be synthesized, in whole or in part, using chemical methods well known in the art (Caruthers et al., Nucleic Acids Res., Symp. Ser., 215-233 (1980)). Alternatively, the probes can be generated, in whole or in part, enzymatically.

The probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Nucleic acid microarrays are preferred. Such microarrays (including nucleic acid chips) are well known in the art (see, for example U.S. Pat. Nos. 5,578,832; 5,861,242; 6,183,698; 6,287,850; 6,291,183; 6,297,018; 6,306,643; and 6,308,170). Probes of the present invention may also be chemically synthesized in situ on chips using photolithographic methods analogous to those used for integrated circuit production (Affymetrix; Lipshutz et al, 1999; U.S. Pat. No. 6,887,665, U.S. Pat. No. 5,143,854; U.S. Pat. No. 6,083,697).

Alternatively, antibody microarrays can be produced. The production of such microarrays is essentially as described in Schweitzer & Kingsmore, 2002; Avseekno et al., 2001; and Huang, 2001.

Similarly, in preferred embodiments the primers of the invention may be used in determining an individual's polymorphism. The presence or absence of specific polymorphisms can be determined in any of a variety of ways as recognized by those skilled in the art.

For example, the nucleotide sequence of at least one nucleic acid sequence which includes at least one polymorphism (or a complementary sequence) can be determined, such as by chain termination methods, ligation methods, hybridization methods or by mass spectrometric methods.

Therefore, in another aspect, the present invention is directed to an isolated nucleic acid molecule suitable for use in detecting a polymorphism selected from the group consisting SEQ ID NOs 2 to 5 of the GMPS gene, said nucleic acid molecule consisting of a nucleotide sequence having about at least 15 contiguous bases of SEQ ID NO 1 or a complementary sequence thereof.

In one embodiment, the nucleic acid molecule consists of a probe having a sequence which binds to the nucleotide sequence which contains at least one polymorphism.

In another embodiment, the nucleic acid molecule consists of a primer having a sequence which binds to the GMPS gene either upstream or downstream of a polymorphism. The primer in a preferred embodiment binds to the GMPS gene sequence upstream or downstream of a polymorphism and up to one base from said polymorphism.

Preferably, the polymorphism is located in the promoter region or in a coding region of the GMPS gene sequence. More preferably, the polymorphism is located in the promoter region at position 692 where a T is replaced by C and/or at position 717 to 718, where a C is inserted between the nucleotides at these positions.

Where the polymorphism is located in a coding region of the GMPS gene sequence, it is preferably in exon 13 at position 62120 where A is replaced by C, and/or at position 62197 where T is replaced by G.

In a still further aspect, the present invention provides an isolated nucleic acid molecule having the sequence of SEQ ID NO:1 and comprising one or more polymorphisms selected from the group comprising SEQ ID NOs 2 to 5, or a fragment, variant or antisense molecule thereof.

The nucleic acid molecule may alternatively comprise peptide nucleic acid (PNA). The nucleic acid may further comprise a detectable label, preferably a fluorescent label. Alternatively, detection may be accomplished by use of radioisotopic labels, or by methods that differentiate mass of reaction products.

In a further preferred embodiment, the nucleic acid molecule contains two polymorphisms comprising SEQ ID NOs: 2 and 3 or SEQ ID NOs: 4 and 5.

Determining the presence or absence of at least two polymorphisms and their relationship on the two gene copies present in an individual can constitute determining a haplotype or haplotypes.

One approach to the detection of the presence or absence of at least one polymorphism may involve contacting a test nucleic acid sequence from an individual with a nucleic acid probe, where the probe is chosen to preferentially hybridize with a form of the nucleic acid sequence containing a complementary base at the polymorphism as compared to hybridization to a form of the nucleic acid sequence having a non-complementary base at the polymorphism, where the hybridization is carried out under selective hybridization conditions, preferably stringent hybridization conditions. Such a nucleic acid hybridization probe may span two or more polymorphisms. Unless otherwise specified, a nucleic acid probe can include one or more nucleic acid analogs, labels or other substituents or moieties so long as the base-pairing function is retained. Preferably, the probe distinguishes at least one polymorphism as identified in any one of SEQ ID No. 2 to 5.

Preferably, the isolated nucleic acid molecule comprises fewer than 10,000 nucleotides, more preferably fewer than 500 nucleotides. The maximum size of 10,000 nucleotides is close to the upper product size limit routinely achievable by polymerase chain reaction (PCR) methods as applied to genomic DNA.

Larger nucleic acid molecules are generally used as primers, whereas smaller molecules are generally used as probes.

It is also preferred in hybridization assays to use probes that comprise a smaller number of nucleotides. At most in such assays you would expect no more than 100 bases, more preferably about 70 bases. The minimum size of 15 nucleotides is a working minimum for an oligonucleotide expected to show specificity in the context of genomic DNA amplification or hybridization.

In preferred embodiments, depending on its use as a primer or a probe, the nucleic acid molecules of the invention have a length in a range between from any one of the above lengths to any other of the above lengths (including endpoints). In a preferred embodiment, the probe is between 15 and 500 nucleotides in length, preferably 15 to 100 nucleotides in length, more preferably 15 to 50 nucleotides in length, and most preferably 15 to 30 nucleotides in length, which has a sequence which corresponds to a portion of the gene identified for aspects above. Preferably the lower limit for the preceding ranges is 17, 20, 22, or 25 nucleotides in length.

In other embodiments, the nucleic acid sequence is 30 to 300 nucleotides in length, or 45 to 200 nucleotides in length, or 45 to 100 nucleotides in length.

A probe should specifically hybridize under selective hybridization conditions to a nucleic acid sequence corresponding to a portion of the GMPS gene. The nucleic acid sequence includes at least one and preferably two or more polymorphisms. Also in preferred embodiments, the probe has a detectable label, preferably a fluorescent label. A variety of other detectable labels are known to those skilled in the art. Such a nucleic acid probe can also include one or more nucleic acid analogs.

The nucleic acid sequence includes at least one polymorphism. Such sequences can, for example, be amplification or ligation products of a sequence which spans or includes a polymorphism in a gene identified herein. Likewise, such a sequence can be a primer, or amplification nucleotide which is able to bind to or extend through a polymorphism in such a gene.

Yet another example is a nucleic acid hybridization probe comprising such a sequence. In such probes, primers, and amplification products, the nucleotide sequence can contain a sequence or site corresponding to a polymorphism, for example, a polymorphism identified herein. Preferably the presence or absence of a particular polymorphism in the heterozygous or homozygous state is indicative of the effectiveness of a method of treatment in an individual.

Likewise, a set of primers or amplification oligonucleotides (e.g., 2,3,4,6,8,10 or even more) are provided adapted for binding to or extending through the GMPS gene. In preferred embodiments the set includes primers or amplification oligonucleotides adapted to bind to or extend through a plurality of sequence polymorphisms in the GMPS gene. The plurality of polymorphisms preferably provides a haplotype. Those skilled in the art are familiar with the use of amplification oligonucleotides (e.g., PCR primers) and the appropriate location, testing and use of such oligonucleotides. In certain embodiments, the oligonucleotides are designed and selected to provide polymorphic-specific amplification.

Genotyping approaches include methods that require allele specific hybridization of primers or probes; allele specific incorporation of nucleotides to primers bound close to or adjacent to the polymorphisms (often referred to as “single base extension”, or “minisequencing”); allele-specific ligation (joining) of oligonucleotides (ligation chain reaction or ligation padlock probes); and allele-specific cleavage of oligonucleotides or PCR products by restriction enzymes (restriction fragment length polymorphisms analysis or RFLP) or by invasive structure specific enzymes (Invader assay).

One type of detection method for use in genotyping can involve the use of detection systems based on electrophoretic separation in agarose or polyacrylamide gels, differential fluorescent or radioactive signals, or detection of differential size or mass of reaction products. These methods variously employ primers that flank, or that lie adjacent to, or that include within their sequence or at their most 3′ position, any of the nucleotides of the invention, more preferably, however:
nucleotide 692 of SEQ ID NO: 1 wherein T is replaced by C (SEQ ID NO:2);
a nucleotide between 717 and 718 of SEQ ID NO: 1 wherein C is inserted (SEQ ID NO:3);
nucleotide 62120 of SEQ ID NO: 1 wherein A is replaced by G (SEQ ID NO:4); and
nucleotide 62197 of SEQ ID NO: 1 wherein T is replaced by G (SEQ ID NO:5), or an antisense molecule thereof.

Other methods for determining polymorphisms are known to art-skilled workers as reviewed by Syvanen and Taylor (2004). One preferred example is the use of mass spectrometric determination of a nucleic acid sequence which is a portion of the GMPS gene or a complementary sequence. Such mass spectrometric methods are known to those skilled in the art, and most of the genotyping assays referred to above could be adapted for the mass spectrometric detection of the GMPS polymorphisms of the invention.

The above method aspects can be facilitated by the provision of kits.

In another aspect, the present invention provides a diagnostic kit for identifying individuals at risk of thiopurine resistance or intolerance based on assessment of the genotypic state of the GMPS gene.

The kit may comprise a probe of the invention. Alternatively a primer of the invention may be employed. Said primer should binds to the GMPS gene or the antisense strand thereof up to a nucleotide positioned one base from a polymorphism.

In a further aspect, the present invention provides a diagnostic kit for identifying individuals at risk of thiopurine resistance or intolerance comprising first and second primers which are complementary to nucleotide sequences of the GMPS gene upstream and downstream, respectfully, of said at least one polymorphism.

Preferably the polymorphism is selected from the group comprising at least one SEQ ID NOs 2 to 5.

In another aspect, the present invention provides a kit for detecting an altered probability of thiopurine resistance or intolerance in an individual, which kit comprises a nucleotide of SEQ ID NO: 1.

In another aspect, the kit comprises a single primer which is substantially complementary to the GMPS gene sequence and binds directly to the nucleotide sequence contrary of at least one polymorphism.

In still a further aspect, the present invention provides a primer suitable for use in detecting a polymorphism selected from the group consisting SEQ ID NOs 2 to 5 of the GMPS gene, said primer consisting of a nucleotide sequence having about at least 15 contiguous bases of SEQ ID NO 1.

The kit is preferably adapted and configured to be suitable for identification of the presence or absence of one or more particular polymorphisms, comprising a nucleic acid sequence corresponding to a portion of a gene. A plurality of polymorphisms may comprise a haplotype or haplotypes.

The kit may also contain a plurality of either or both of such probes and/or primers, e.g., 2, 3, 4, 5, 6, or more of such probes and/or primers. Preferably the plurality of probes and/or primers are adapted to provide detection of a plurality of different sequence polymorphisms in a gene or plurality of genes, e.g., in 2, 3, 4, 5, or more genes or to amplify and/or sequence a nucleic acid sequence including at least one polymorphism in a gene or genes.

Preferably one or more of the polymorphism or polymorphisms to be detected are correlated with variability in a treatment response or tolerance, and are preferably indicative of an effective response to a treatment.

In preferred embodiments, the kit contains components (e.g., probes and/or primers) adapted or useful for detection of a plurality of polymorphisms of GMPS indicative of the effectiveness of at least one treatment, preferably of a plurality of different treatments for a particular disease or condition. In the currently preferred embodiment, the condition is inflammatory bowel disease (IBD).

It may also be desirable to provide a kit containing components adapted or useful to allow detection of a plurality of polymorphisms indicative of the effectiveness of a treatment or treatment against a plurality of diseases. The kit may also optionally contain other components, preferably other components adapted for identifying the presence of a particular polymorphism or polymorphisms. Such additional components can, for example, independently include a buffer or buffers, e.g., amplification buffers and hybridization buffers, which may be in liquid or dry form, a DNA polymerase, e.g., a polymerase suitable for carrying out PCR (e.g., a thermostable DNA polymerase), a DNA ligase, e.g. a DNA ligase suitable for performing the ligase chain reaction, specialised probes (such as padlock probes), and deoxynucleoside triphosphates (dNTPs), dideoxynucleoside triphosphates (ddNTPs) or ribonucleotide triphosphates.

Preferably, the kit comprises several oligonucleotides that will hybridize specifically to the GMPS gene. These oligonucleotides will enable specific amplification of GMPS nucleotides from human genomic DNA or cDNA template, using PCR. Most preferably, these oligonucleotides will also enable specific genotyping of the GMPS gene by acting as primers, probes, or ligation substrates that enable differentiation of polymorphic alleles. Alternatively, these oligonucleotides may be suitable for use in emerging methods that do not depend on prior amplification of the starting DNA, such as Invader assays and ligation-based detection methods. Preferably the oligonucleotides or other kit components will include a detectable label, e.g., a fluorescent label, enzyme label, light scattering label, mass label, or other label. Alternatively, detection may be achieved by RFLP methods (as described in the Example 1 and 2). In addition, the kit may include a plurality of different nucleic acid sequences allowing detection of nucleic acid sequences or gene products corresponding to different polymorphisms or haplotypes of GMPS. Preferably the kit is arranged to provide polymorphism detection for a plurality of polymorphisms in GMPS which correlate with the effectiveness of one or more treatments of one or more diseases, which is preferably a polymorphism as described herein.

The kit may also optionally contain instructions for use, which can include a listing of the polymorphisms correlating with a particular treatment or treatments for a disease or diseases and/or a statement or listing of the diseases for which a particular polymorphism or polymorphisms correlates with a treatment efficacy and/or safety.

Preferably the kit components are selected to allow detection of a polymorphism described herein, and/or detection of a polymorphism indicative of a treatment, or a polymorphism that contra-indicates a treatment.

Preferably the kit components may include samples of “control” DNA, constituting genomic DNA from individuals with different alleles of each of the indicated polymorphisms. This will enable quality control of the assay when applied in different laboratories.

The methods of the invention are particularly useful for detecting polymorphisms associated with thiopurine resistance or intolerance in patients suffering from IBD, or subtypes of IBD, which has been classified into the broad categories of Crohn's disease and ulcerative colitis. Crohn's disease (regional enteritis) is a disease of chronic inflammation that can involve any part of the gastrointestinal tract.

Commonly, the distal portion of the small intestine (ileum) and cecum are affected. In other cases, the disease is confined to the small intestine, colon oranorectal region. Crohn's disease occasionally involves the duodenum and stomach, and more rarely the esophagus and oral cavity. The most frequent symptoms of Crohn's disease are abdominal pain, diarrhea and recurrent fever, and this disease also can be associated with intestinal obstruction (strictures) or fistula, which is an abnormal passage between diseased loops of bowel. Crohn's disease further can be associated with complications such as inflammation of the eye, joints and skin; liver disease; kidney stones or amyloidosis.

The pathology of Crohn's disease includes transmural inflammation, involving all layers of the bowel wall. Thickening and edema, for example, typically appear throughout the bowel wall, with fibrosis also present in long-standing disease. Furthermore, the inflammation characteristic of Crohn's disease also is discontinuous in that segments of inflamed tissue, known as “skip lesions”, are separated by apparently normal intestine. Linear ulcerations, edema, and inflammation of the intervening tissue lead to a “cobblestone” appearance of the intestinal mucosa, which is distinctive of Crohn's disease. A hallmark of Crohn's disease is the presence of discrete aggregations of inflammatory cells, known as granulomas, which are generally found in the submucosa (Rubin and Farber, (1994).

The inflammatory bowel disease ulcerative colitis (UC) is a disease of the large intestine characterized by chronic diarrhea with cramping abdominal pain, rectal bleeding, and loose discharges of blood, pus and mucus. The manifestations of ulcerative colitis vary widely. A pattern of exacerbations and remissions typifies the clinical course of most UC patients (70%), although continuous symptoms without remission are present in some patients with ulcerative colitis. Local and systemic complications of UC include arthritis, eye inflammation such as uveitis, skin ulcers and liver disease. In addition, ulcerative colitis, and especially long-standing, extensive disease, is associated with an increased risk of colon carcinoma.

UC generally is a diffuse disease that usually extends from the most distal part of the rectum for a variable distance proximally. Sparing of the rectum or involvement of the right side (proximal portion) of the colon alone is unusual in UC. The inflammatory process of UC is limited to the colon and is distinguished by a superficial inflammation of the mucosa that generally spares the deeper layers of the bowel wall. (Rubin and Farber, 1994).

The methods of the invention are useful for determining thiopurine intolerance or resistance in a variety of subjects, including patients having inflammatory bowel disease or leukemia, and organ or allograft transplant recipient.

The methods of the invention may also be performed in conjunction with an analysis of one or more risk factors such as but not limited to age, weight, sex, family history of events such as thiopurine intolerance or resistance, and thiopurine methyltransferase (TPMT) activity. Intermediate or low TPMT activity is associated with susceptibility to the toxic side effects of thiopurine therapy (Present et al, 1989; EP 1285085).

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### Example 1

### Protocol for the PCR-RFLP Assay Used to Detect Promoter SNPs

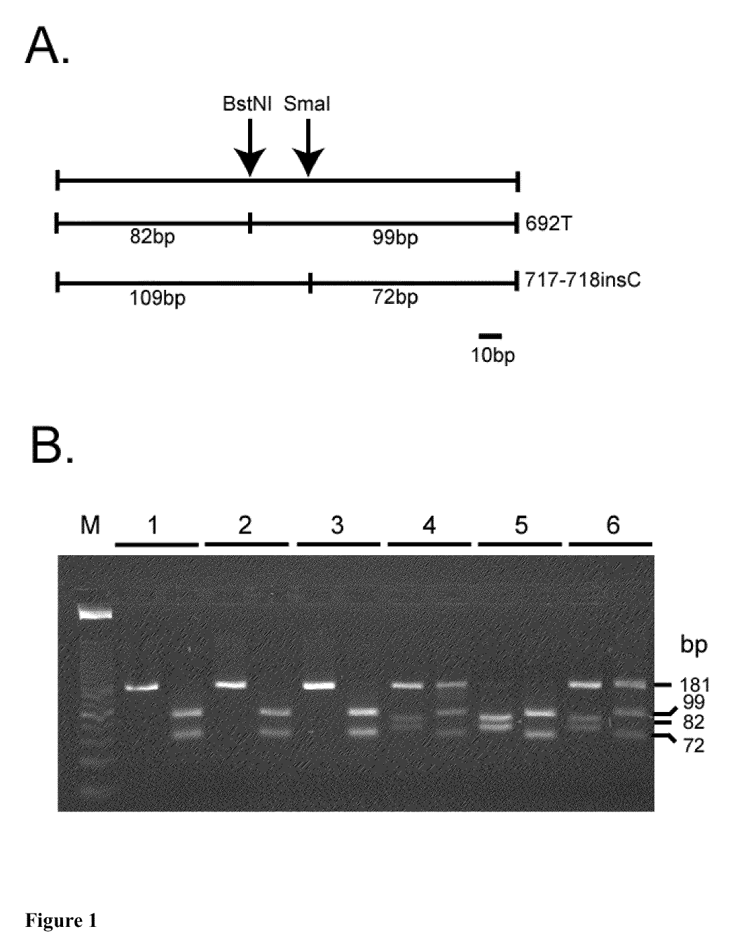

Separate PCR-RFLP assays were applied for each promoter polymorphism. FIG. 1A illustrates the 181 by fragment generated by PCR which encompasses both GMPS promoter SNPs, indicating the restriction enzyme sites for BstNI (specific for 692T) and SmaI (specific for 717 to 718 insC).

The PCR was performed in a total volume of 25 μl containing 200 μM of each dNTP, 1.5 mM MgCl₂, 1× Q-Solution (QIAGEN Pty Ltd, Victoria, Australia), 0.5 μM of primers GMPSinsCf (5′-CGCCAGCCTCCCGAAGTCATCCAAGGT-3′) and GMPSInsr (5′-GCCCTTGGGAGGAGGAGCCC-3′), 1U of Taq DNA Polymerase (Roche Diagnostics GmbH, Mannheim, Germany) and ˜100 ng of DNA. Thermal cycling conditions for this PCR were as follows: 95° C. for 15 minutes; followed by 35 cycles of 94° C. for 1 minutes, 62° C. for 30 seconds, and 72° C. for 50 seconds; and a final extension of 72° C. for 2 minutes. Separate 3W aliquots of the PCR were then digested with the restriction endonucleases SmaI and BstNI. The SNPs 692C>T and 717-718insC create recognition sites for BstNl and SmaI, respectively.

With reference to FIG. 1B, all digested PCR products were resolved on 3% agarose and sized using a 25 bp DNA ladder (M). The left hand lane of each paired sample contains the BstNI digestion products, the right hand lane the SmaI digestion products. Samples 1, 2, & 3 are homozygous for 692C and are negative for 717-718insC; samples 4 & 6 are heterozygous for both SNPs; and sample 5 is homozygous for 692T and 717-718insC.

These assays were used to determine whether the frequency of 692T>C or 717-718insC varied between individuals that exhibited thiopurine drug resistance, and individuals that exhibited favourable 6-TGN:6-MMPR ratios. In the first instance, six inflammatory bowel disease (IBD) individuals with unfavourable 6-TGN:6-MMPR ratios, and eighteen IBD individuals with therapeutic levels of 6-TGNs were genotyped for the GMPS promoter SNPs (see Table 1 below).

TABLE 1
Frequency of GMPS promoter polymorphisms
in IBD individuals and healthy volunteers

| | IBD non-responders | IBD responders | Controls |
|---|---|---|---|
| Variant | (n = 6) | (n = 18) | (n = 170) |
| 692T > C | | | |
| C/C | 0 | 9 (50%) | 68 (40%) |
| C/T | 3 (50%) | 7 (38%) | 82 (48%) |
| T/T | 3 (50%) | 2 (11%) | 20 (12%) |
| 717-718InsC | | | |
| —/— | 2 (33.3%) | 1 (5.6%) | 11 (6.5%) |
| Ins/— | 4 (66.7%) | 4 (22.2%) | 52 (30.6%) |
| Ins/Ins | 0 | 13 (72.2%) | 107 (62.9%) |

Each SNP was detected in a heterozygous state in both individual groups. No 692C or 717-718insC homozygotes were found in the IBD individuals that exhibited thiopurine resistance. In contrast, the occurrence of 717-718insC homozygotes within the IBD responders was 72.2%. Subsequent genotyping of 170 healthy New Zealand Caucasians also found a high incidence of individuals that carried two copies of 692C and 717-718insC.

### Example 2

### Protocol for the PCR-RFLP Assays Used to Detect Exon 13 SNPs

Separate PCR-RFLP assays were applied to detect the two nonsynonymous exon 13 SNPs (62120A>C and 62197T>G). The first SNP (62120A>C) abolishes a BsaAI recognition site, whereas the second exon 13 SNP (62197T>G) creates a BslI recognition site.

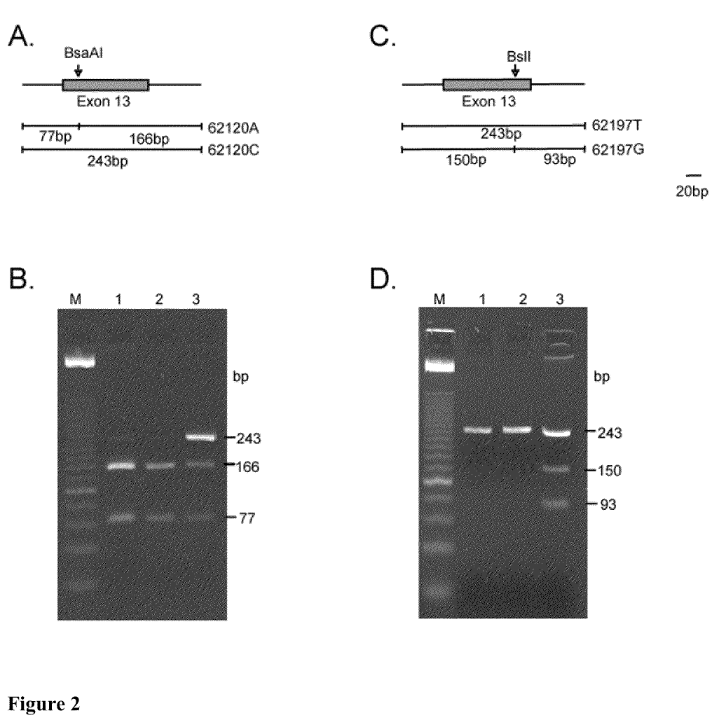

FIG. 2A illustrates the 243 bp fragment generated by PCR which encompasses both GMPS exon 13 SNPs, indicating the restriction enzyme site for BsaAI (specific for 62120A). FIG. 2C illustrates the 243 bp fragment generated by PCR which encompasses both GMPS exon 13 SNPs, indicating the restriction enzyme site for BslI (specific for 62197G)

Amplification of exon 13 was performed in 25 μl containing 200 μM of each dNTP, 2.5 mM MgCl₂, 0.5 μM of the forward primer GMPSex13f (5′-AACTGGTGTATCTTTTGACTATTA-3′) and the reverse primer GMPSex13r (5% CATTAATTGAAAGCCCTTAAGAAAT-3′), 1U of HotMaster™ DNA Tag Polymerase (Eppendorf, Hamburg, Germany), and ˜100 ng genomic DNA. Thermocycling conditions were as follows: 94° C. for 2 minutes; followed by 35 cycles of 94° C. for 30 seconds, 56° C. for 10 seconds, 68° C. for 40 seconds; and a final extension step of 68° C. for 1 minute.

Three microlitres of PCR product were incubated in a total volume of 10 μl containing 1 unit of BsaAI or BslI (New England Biolabs, MA, USA), and I_{A}A of the appropriate commercial buffer. After two hours incubation, the digested PCR products were resolved on 3% 1×TBE-LE agarose adjacent to a 25 bp DNA ladder (M). FIG. 2B illustrates the agarose gel resulting from electrophoresis of the BsaAI restriction digestion products, and FIG. 2D illustrates the agarose gel resulting from electrophoresis of the BslI restriction digestion products. Table 2, below sets out these results.

TABLE 2
Frequencies of the GMPS exon 13 SNPs in
unaffected controls and IBD individuals

| | SNP Frequency % (number of GMPS | |
|---|---|---|
| | alleles) | |
| Sample | 62120C | 62197G |
| IBD individuals (↓6-TGN:↑6- | 50 (2/4) | 25 (1/4) |
| MMP)^{a} | | |
| IBD individuals (↑6-TGN:↓6- | 0 (0/188) | 0.0 (0/188) |
| MMP)^{b} | | |
| Controls | 2.2 (4/182) | 0.5 (1/182) |

^{a}IBD individuals with an unfavourable 6-TGN:6-MMP ratio
^{b}IBD individuals with a favourable 6-TGN:6-MMP ratio

It is not the intention to limit the scope of the invention to the abovementioned examples only. As would be appreciated by a skilled person in the art, many variations are possible without departing from the scope of the invention as set out in the accompanying claims.

### REFERENCES

Pegram, L. D.; Megonigal, M. D.; Lange, B. J.; Nowell, P. C.; Rowley, J. D.; Rappaport, E. F.; Felix, C. A.: t(3; 11) translocation in treatment-related acute myeloid leukemia fuses MLL with the GMPS (guanosine 5-prime monophosphate synthetase) gene, Blood 96: 4360-4362, 2000. PubMed ID: 11110714
Kwok, P. Y. (2001). Methods for genotyping single nucleotide polymorphisms. Annu Rev Genomics Hum Genet 2, 235-258.
Syvanen, A. C., and Taylor, G. R. (2004). Approaches for analyzing human mutations and nucleotide sequence variation: a report from the Seventh International Mutation Detection meeting, 2003. Hum Mutat 23, 401-405.
Reich DE et al (2001). Linkage disequilibrium in the human genome, Nature 2001, 411:199-204.
Caruthers et al (1980). Nucleic Acids Res., Symp. Ser., 215-233.
Schweitzer & Kingsmore (2002). Measuring proteins on microarrays, Curr Opin Biotechnol, 13(1): 14-9.
Avseekno et al (2001). Immobilization of proteins in immunochemical microarrays fabricated by electrospray deposition, Anal Chem 15; 73(24): 6047-52.
Huang (2001). Detection of multiple proteins in an antibody-based protein microarray system, Immunol Methods, 1; 255(1-2): 1-13.
Rubin & Farber (1994. Pathology (Second Edition) Philadelphia: J.B. Lippincott Company.
Present et al (1989). Annals of Internal Medicine 111: 641-649.
Lipshutz R J, Fodor S P, Gingeros T R, Lockhart D J: High density synthetic oligonucleotide arrays. Nature Genetics 1999, 21:20-24.

All references and citations in this list and throughout the specification, including patent specifications, are hereby incorporated in their entirety.

### INDUSTRIAL APPLICATIONS

The present invention is useful for detecting individuals suffering from diseases that are treated using thiopurine therapy, but who are intolerant or resistant to such therapy. Such diseases include acute lymphoblastic leukaemia, inflammatory bowel disease, complications associated with solid organ transplantation, rheumatoid arthritis, dermatological conditions and autoimmune conditions.

1. A method for screening individuals for the presence or absence of one or more polymorphisms associated with the risk of thiopurine resistance or intolerance, which method includes the step of determining the genotypic state of the individual with respect to the GMPS gene.

2. A method as claimed in claim 1, wherein the genotypic state is determined with respect to DNA, or with respect to mRNA if a polymorphism is in the coding region, obtained from said individual, by direct or indirect methods.

3. A method as claimed in claim 2, wherein a biological sample containing DNA is obtained from an individual and the genotypic state of the GMPS gene assessed for the presence of at least one nucleotide difference from the nucleotide sequence encoding GMPS (SEQ ID NO: 1), either by direct or indirect methods.

4. A method as claimed in claim 1, wherein the genotypic state is determined by the presence of one or more polymorphisms selected from SEQ ID NOs 2 and 5, either by direct or indirect methods.

5. A method as claimed in claim 1, w herein the polymorphism is located in the promoter region or in a coding region of the GMPS gene sequences.

6. A method as claimed in claim 5, wherein the polymorphism is located in the promoter region at position 692 where a T is replaced by C and/or at position 717 to 718, where a C is inserted between the nucleotides at these positions.

7. A method as claimed in claim 5, wherein the polymorphism is located in a coding region of the GMPS gene sequence, in exon 13 at position 62120 where A is replaced by C, and/or at position 62197 where T is replaced by G.

8. A method of identifying an individual at risk of thiopurine resistance or intolerance, said method comprising:
obtaining a biological sample containing nucleic acids from said individual and identifying a polymorphism selected from the group consisting of SEQ ID NOs 2 to 5 of the GMPS gene, wherein the presence of said polymorphism is associated with a risk of thiopurine resistance or intolerance.

9. A method as claimed in claim 8, wherein the polymorphism located in the promoter region or in a coding region of the GMPS gene sequence.

10. A method as claimed in claim 9, wherein the polymorphism is located in the promoter region at position 692 where a T is replaced by C and/or at position 717 to 718, where a C is inserted between the nucleotides at these positions.

11. A method as claimed in claim 9, wherein the polymorphism is located in a coding region of the GMPS gene sequence, in exon 13 at position 62120 where A is replaced by C, and/or at position 62197 where T is replaced by G.

12. An isolated nucleic acid molecule when used in detecting a polymorphism selected from the group consisting of SEQ ID NOs 2 to 5 of the GMPS gene, said nucleic acid molecule consisting of a nucleotide sequence having about at least 15 contiguous bases of SEQ ID NO 1 or a complementary sequence thereof.

13. An isolated nucleic acid molecule as claimed in claim 12, consisting of a probe having a sequence which binds to the nucleotide sequence which contains at least one polymorphism.

14. An isolated nucleic acid molecule as claimed in claim 12, consisting of a primer having a sequence which binds to the GMPS gene either upstream or downstream of one or more said polymorphisms.

15. An isolated nucleic acid molecule as claimed in claim 14, wherein the primer binds to the GMPS gene sequence up to one base upstream or downstream from one or more of said polymorphisms.

16. An isolated nucleic acid molecule as claimed in claim 12, wherein the polymorphism is located in the promoter region or in a coding region of the GMPS gene sequence.

17. An isolated nucleic acid as claimed in claim 16, wherein the polymorphism is located in the promoter region at position 692 where a T is replaced by C and/or at position 717 to 718, where a C is inserted between the nucleotides at these positions.

18. An isolated nucleic acid molecule as claimed in claim 12, wherein the polymorphism is located in a coding region of the GMPS gene sequence, in exon 13 at position 62120 where A is replaced by C, and/or at position 62197 where T is replaced by G.

19. An isolated nucleic acid molecule having the sequence of SEQ ID NO: 1 and comprising one or more polymorphisms selected from the group comprising SEQ ID NOs 2 to 5, or a fragment, variant or antisense molecule thereof.

20. A nucleic acid molecule as claimed in claim 12, comprising a peptide nucleic acid (PNA).

21. A nucleic acid molecule as claimed in claim 12, further comprising a detectable label.

22. A nucleic acid molecule as claimed in claim 21, wherein the detectable label is a fluorescent label.

23. An isolated nucleic acid molecule as claimed in claim 21, wherein the detectable label is a radioisotopic label.

24. An isolated nucleic acid molecule as claimed in claim 12, wherein the nucleic acid molecule contains two polymorphisms comprising SEQ ID NOs: 2 and 3 or SEQ ID NOs: 4 and 5.

25. A diagnostic kit for identifying individuals at risk of thiopurine resistance or intolerance based on assessment of the genotypic state of the GMPS gene, wherein said kit comprises a probe as claimed in claim 13.

26. A diagnostic kit for identifying individuals at risk of thiopurine resistance or intolerance based on assessment of the genotypic state of the GMPS gene, wherein said kit comprises a primer as claimed in claim 14.

27. A diagnostic kit for identifying individuals at risk of thiopurine resistance or intolerance comprising first and second primers which are complementary to nucleotide sequences of the GMPS gene or the antisense strand thereof upstream and downstream, respectively, of at least one polymorphism selected from the group consisting of SEQ ID NOS: 2 to 5.

28. A nucleic acid molecule as claimed in claim 19, comprising a peptide nucleic acid (PNA).

29. A nucleic acid molecule as claimed in claim 19, further comprising a detectable label.

30. A nucleic acid molecule as claimed in claim 29, wherein the detectable label is a fluorescent label.

31. An isolated nucleic acid molecule as claimed in claim 29, wherein the detectable label is a radioisotopic label.

32. An isolated nucleic acid molecule as claimed in claim 19, wherein the nucleic acid molecule contains two polymorphisms comprising SEQ ID NOs: 2 and 3 or SEQ ID NOs: 4 and 5.
